(19) United States
(12) Patent Application Publication
(10) Pub. No.: US 2025/0011853 A1
(43) Pub. Date: Jan. 9, 2025

(54) METHOD OF EXAMINING A NUCLEIC ACID AMPLIFICATION PRODUCT

(71) Applicant: QIAGEN GmbH, Hilden (DE)

(72) Inventor: Ronny KELLNER, Bad Honnef (DE)

(21) Appl. No.: 18/697,458

(22) PCT Filed: Sep. 30, 2022
(86) PCT No.: PCT/EP2022/077356

§ 371 Date: Mar. 30, 2024

(30) Foreign Application Priority Data
Oct. 1, 2021 (EP) 21200524.3

(51) Int. Cl. C12Q 1/6853; C12Q 1/6806; C12Q 1/686

(52) U.S. Cl. CPC C12Q 1/6853; C12Q 1/6806; C12Q 1/686

(57) ABSTRACT

The invention relates to a method for examining a nucleic acid amplification product, comprising the steps of a. providing an amplification product stemming from a preferably exponential amplification comprising i. an upstream and downstream primer region as well as optionally and preferably, ii. an amplified target region between said upstream and downstream primer region, b. examining the amplification product by amplifying said amplification product in a PCR reaction, wherein i. the upstream and downstream PCR primers in said PCR reaction bind in the upstream and downstream primer region of the amplification product, ii. a first and a second oligonucleotide probe are placed downstream of the first and second PCR primers and within the upstream and the downstream primer region, c. performing a PCR reaction and examining the newly formed amplification product, wherein the upstream and downstream primer regions of step a.i.) have a length of under 35 nucleotides and over 20 nucleotides.

### BACKGROUND

Next-generation sequencing technologies, such as pyrosequencing, sequencing by synthesis, and sequencing by oligonucleotide ligation and detection, overcome the major limitations of the first-generation approach. Sequencing reactions can be performed in parallel with a very large number of different samples (templates) immobilized in an array in the same flow cell. The density of samples per unit area can be very high, and the total number of samples can be increased by enlarging the array. The samples can be exposed to a series of sequencing reagents in parallel in a shared fluid volume inside the flow cell. Also, the samples in the array can be monitored with a camera to record sequence data from all of the samples in real time as the sequencing reactions proceed in parallel with cyclical exposure to reagents passing through the flow cell; See WO 2013/019751.

Next-generation technologies currently on the market rely on in vitro libraries having a particular construction. The various fragments to be sequenced are each flanked by adapters to form library members. The adapters provide primer binding sites for clonal amplification of each library member on a support, such as on a flat surface or beads. The adapters can introduce binding sites that enable amplification of all members of the library with the same primer or pair of adapter-specific primers. Also, one or both of the adapters can provide a binding site for a sequencing primer. Furthermore, an adapter can introduce a library-specific index sequence that permits members of different libraries to be pooled and sequenced together in the same flow cell, without losing track of the library of origin for each member. A set of libraries can be constructed in parallel, such as in different wells of a multi-well plate, from different nucleic acid samples. However, despite the best efforts to achieve uniform reaction conditions among the wells, the concentration and quality of the libraries can vary widely. With the sequencing capacity of NGS instrumentation continuing to rise, researchers are able to pool more samples, or libraries, into a single sequencing run, greatly reducing the per-sample cost of sequencing. However, NGS library concentrations can vary widely, based on the amount and quality of nucleic acid sample inputs, as well as the target enrichment method that is used. To ensure that each pooled library is sequenced to the desired depth, NGS libraries must be carefully quantified and normalized so that each sample achieves the required number of reads. Common library quantification methods include fluorometric spectroscopy and quantitative PCR (qPCR). While both methods provide relatively accurate measurement of library concentration, there are assay specific considerations associated with these techniques. In this document, we provide a comparison of two library quantification technologies: the Invitrogen™ Qubit™ dsDNA HS Assay Kit and the Invitrogen™ Collibri™ Library Quantification Kit, which utilize the Invitrogen™ Qubit™ Fluorometer or qPCR, respectively.

### SUMMARY OF THE INVENTION

The present disclosure provides methods of characterizing a nucleic acid library by using digital amplification assay.

The invention thus relates to a method for examining a nucleic acid amplification product, comprising the steps of
a. providing an amplification product stemming from a linear or exponential amplification comprising
   i. an upstream and a downstream primer region and an upstream and a downstream primer binding said upstream and said downstream primer region, respectively, as well as optionally and preferably,
   ii. an amplified target region between said upstream and downstream primer region,
b. examining the amplification product by amplifying said amplification product in a PCR reaction, wherein
   i. the upstream and downstream PCR primers in said PCR reaction bind in the upstream and downstream primer region of the amplification product,
   ii. a first and a second oligonucleotide probe are placed downstream of the first and second PCR primers and within the upstream and the downstream primer region,
c. performing a PCR reaction and examining the newly formed amplification product, wherein the upstream and downstream primer regions of step a.i.) have a length of under 35 nucleotides and over 20 nucleotides.

Preferably the amplification is exponential.

An exemplary method of library characterization is provided. However, the invention may be applied to various kinds of amplification products. In the method, a nucleic acid library may be obtained first. The library may include members each having a first adapter region and a second adapter region. Characterization of these libraries has been difficult to standardize as the adapters differ in length often or are very short. That makes assay development very difficult.

Examining is preferably, i) quantifying the amount of amplification product, or ii) checking whether the amplification product contains the desired insert, or iii) both.

At least a subset of the members may have an insert disposed between the first and second adapter regions. At least a portion of the library may be divided into partitions. A digital assay may be performed on the partitions with an adapter region probe to generate data indicating whether a library member is present in each partition. A characteristic of the library may be determined based on the data. Another exemplary method of library characterization is provided. In the method, a nucleic acid library may be obtained. The library may include members each having a first constant region and a second constant region. At least a subset of the members may have a variable region disposed between the first and second constant regions. Partitions containing members of the library at limiting dilution may be formed. Members of the library may be amplified in the partitions using a primer for each constant region. Amplification data may be collected from a constant region probe in the partitions. A level of members of the library may be determined based on the amplification data.

Library characterization before sequencing can be problematic. Only properly formed library members containing both adapters in the correct relative orientation produce clonal populations that can be interrogated reliably by sequencing. Malformed members in the library, such as members flanked by two copies of only one of the adapters, can be difficult to distinguish from those that are well-formed. However, the malformed members generally cannot be amplified on a support, a prerequisite to sequence acquisition, or do not have a binding site for the sequencing primer, or both. As a result, malformed members can take up space and consume reagents and can reduce the amount of useful sequence information produced by a next-generation sequencing run, in direct proportion to the fraction of malformed members in the library; See WO 2013/019751.

The present method solved these problems. FIG. 1 shows the principal set-up. It is preferable in the present method if the amplification step b) is a digital PCR amplification. In this preferred embodiment the template is subdivided into partitions or droplets. A digital assay may be performed on the partitions with an adapter region probe to generate data indicating whether a library member is present in each partition. A characteristic of the library or template may be determined based on this data.

Digital polymerase chain reaction (digital PCR, DigitalPCR, dPCR, or dePCR) is a biotechnological refinement of conventional polymerase chain reaction methods that can be used to directly quantify and clonally amplify nucleic acids strands including DNA, cDNA, or RNA. The key difference between dPCR and traditional PCR lies in the method of measuring nucleic acids amounts, with the former being a more precise method than PCR, though also more prone to error in the hands of inexperienced users. A “digital” measurement quantitatively and discretely measures a certain variable, whereas an “analog” measurement extrapolates certain measurements based on measured patterns. PCR carries out one reaction per single sample. dPCR also carries out a single reaction within a sample, however the sample is separated into a large number of partitions and the reaction is carried out in each partition individually. This separation allows a more reliable collection and sensitive measurement of nucleic acid amounts. The method has been demonstrated as useful for studying variations in gene sequences—such as copy number variants and point mutations—and it is routinely used for clonal amplification of samples for next-generation sequencing.

The present invention is characterized by the fact that the primers are very short when compared to standard primers. Likewise, the probes used are very short when compared to standard probes. This brings with it problems concerning melting temperatures and melting curves.

Current methods for NGS library quantification are electrophoresis, quantitative real-time PCR, and more recently next-generation sequencing and digital PCR. Among them SYBR Green-based assays using two primers each specific to target one of the two adapters are used by most customers. A more recent development are TaqMan probe based assays using two primers and two probes each specific for one of the two ligated adapters in the library. In contrast to SYBR Green-based assays they are more accurate as unspecific amplicons do not produce a fluorescence signal. The probes of Taqman probe-based assays that are currently on the market are intended and only work for the quantification of NGS libraries from specific preparation kits. This indicates a probe design that targets the variable part of the adapters. Also, it requires the use of multiple assays for quantification of different Illumina libraries.

On a preferred embodiment of the invention the present method overcomes this restriction. Here, the probes lie in the conserved adapter regions P5 and P7. In a further preferred embodiment this is combined with nanoplate-based digital PCR on the QIAcuity platform.

Preferably both probes are designed to target either the same strand or opposite strands and they preferably carry different fluorophores or another labelling system. Even more preferred, said labelling system comprises rare earth cryptates or rare earth chelates in combination with a fluorescence dye or chemiluminescence dye, in particular a dye of the cyanine type. In the context of the present invention, fluorophores comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

Preferably the oligonucleotide probes of the present invention are hydrolysis probes.

Hydrolysis probes are a popular detection chemistry for monitoring sequence-specific amplification in PCR or digital PCR (dPCR). Just like with SYBR Green dye, signal detection is achieved through monitoring an increase in fluorescence as the reaction proceeds. But, the fluorescent signal in TaqMan™ chemistry is dependent on probe hydrolysis, rather than hybridization, hence the name “hydrolysis probes”. In the hydrolysis probes set up, there are two primers and a probe. The probe, also designed complementary to the target, contains the fluorophore and quencher on either end; See also FIG. 1.

During the amplification process, the probe binds to the specific target sequence during the annealing step. Because of the proximity between the donor (fluorophore) and acceptor (quencher) on the probe, there is no fluorescence. During the extension step, the 5′-3′ exonuclease activity of the polymerase hydrolyses the probe relieving the fluorophore from quenching effects and fluorescence is read by the detector.

With this design, a single target DNA that has been linked to both adapters will produce a double fluorescence signal that, after endpoint amplification, can be detected and quantified in digital PCR. As the target regions in principle can be relatively short, primers and probes preferably comprise LNAs to compensate for reduced binding affinity.

The present invention therefore for the first time provides for a single assay that can be used to simultaneously quantify different types of libraries in one reaction. The combination of the assay with the QIAcuity platform and the QIAcuity probe mastermix chemistry enables taking advantage of the precision and accuracy of absolute quantification of digital PCR for NGS library quantification. The assay designs will also work in combination with qPCR.

The present invention also relates to a nucleic acid amplification composition comprising:
a. at least two primers having a length of between 10 and 22 nucleotides and optionally comprising one or more locked nucleic acid nucleotide analogues or another nucleotide analogue that increase template binding strength and,
b. at least two oligonucleotide probes having a length of between 8 and 17 nucleotides and optionally comprising one or more locked nucleic acid nucleotide analogues or another nucleotide analogue that increase template binding strength.

The present invention also relates to the use of the inventive composition for the analysis of a nucleic acid library.

The present invention also relates to a kit comprising:
a. at least two primers which have a length of between 10 and 22 nucleotides and optionally comprising one or more locked nucleic acid nucleotide analogues or another nucleotide analogue that increase template binding strength and,
b. at least two oligonucleotide probes having a length of between 8 and 17 nucleotides and optionally comprising one or more locked nucleic acid nucleotide analogues or another nucleotide analogue that increase template binding strength.

Analysis or examination herein refers to, identifying whether or not amplification has taken place, identifying whether or not the target sequence lies between the primer regions and optionally, has the right length, identifying the amount of amplification product with a correct target sequence. Preferably in the method of the invention precise quantification of the amplification product is desired.

Herein, a “primer region” (or priming region) is a region within a nucleic acid molecule that allows for binding of an oligonucleotide primer. This region is typically between 15 and 40 nucleotides in length, more preferably between 20 and 35 nucleotides in length and most preferably between 15 and 25 nucleotides in length. The sequence therein will be the reverse complement of the sequence of the primer that binds said “primer region”. Of course, the sequence must not be 100% the reverse complement. It might vary a bit provided the respective primer can bind.

Herein, an “amplification product” is a nucleic acid product, either double stranded or single stranded stemming from a nucleic acid amplification reaction. Such a reaction may be an isothermal or non-isothermal amplification. It will typically comprise a sequence stemming from the one or more primers used to amplify the target nucleic acid. Preferably herein this is double stranded and stems from PCR.

### DETAILED DESCRIPTION OF THE INVENTION

Depending on the sequencing goal and the amount of output from the different types of sequencing instruments, the library used for sequencing consists of either a single library or a pool of sub-libraries. In both cases an accurate quantification is required to achieve optimal cluster density for the sequencing run and to prevent over- or undersaturation of the flow cell. Equimolar pooling of sub-libraries prior sequencing requires an additional step of accurate quantification.

The quantity and quality of prepared NGS DNA libraries is determined by the use of different methods. This includes UV absorption (e.g., Nanodrop); intercalating dyes (e.g., QuBit; Invitrogen, SYBR Green); electrophoresis coupled with intercalating dyes (Agilent Bioanalyzer), 5′ hydrolysis probes (e.g., TaqMan®) coupled with real-time quantitative PCR (qPCR; Kapa Biosystem), NGS library quantification (MiSeq) or droplet digital PCR (Bio-Rad). The major disadvantage of DNA quantification using intercalating dyes is the unspecific binding of the dye to any double stranded DNA. In case of the presence of non-target DNA fragments in the library to which the dye will bind this leads to an over quantification of target library DNAs.

Digital PCR provides certain advantages over other commonly used DNA quantification strategies (e.g. QuBit and qPCR). With absolute quantification of single molecules digital PCR uses less amounts of input DNA and does not require the back-calculation of the library against an average size determined by a Bioanalyzer assay. This makes dPCR-based quantification less time- and reagent-consuming while still providing similar sensitivity and accuracy as qPCR. Also it is more sensitive compared to quantifications using QuBit and PicoGreen. Another advantage of digital PCR is that amplification occurs in separate partitions. Even if amplification efficiency differs from amplicon to amplicon or extraction to extraction, sufficient amplicon will be generated in a dPCR run to determine whether a target was present. Digital PCR will therefore provide binary values (present, even if poorly amplified; or absent) for the template of interest, that, corrected for the possibility of having one or more template molecules per partition using Poisson statistics, reveals absolute quantification of molecules/μl.

Compared to droplet digital PCR (ddPCR), as provided by BioRad, dPCR on the QIAcuity system uses 96 and 24 well nanoplates in standard format that are suitable for usage in fully automated workflows. Further, the ddPCR™ Library Quantification Kit from BioRad is restricted to the quantification of Illumina TruSeq libraries. Similarly, the qPCR NGS library quantification assays from Thermo Fisher Scientific are restricted to individual Illumina library types, e.g. the Illumina® Nextera Library Quantification Assay or the Ilumina® TruSeq DNA/RNA Library Quantification Assay.

The invention relates to a method for examining a nucleic acid amplification product, comprising the steps of
a. providing an amplification product stemming from a linear or exponential amplification comprising
   i. an upstream and a downstream primer region and an upstream and a downstream primer binding said upstream and said downstream primer region, respectively, as well as optionally and preferably,
   ii. an amplified target region between said upstream and downstream primer region,
b. examining the amplification product by amplifying said amplification product in a PCR reaction, wherein
   i. the upstream and downstream PCR primers in said PCR reaction bind in the upstream and downstream primer region of the amplification product,
   ii. a first and a second oligonucleotide probe are placed downstream of the first and second PCR primers and within the upstream and the downstream primer region,
c. performing a PCR reaction, preferably a digital PCR, and examining the newly formed amplification product, wherein the upstream and downstream primer regions of step a.i.) have a length of under 35 nucleotides and over 20 nucleotides.

In a preferred embodiment said target region is between 15 and 35 nucleotides in length, between 16 and 34 nucleotides in length, between 17 and 33 nucleotides in length, between 18 and 32 nucleotides in length, between 19 and 31 nucleotides in length, between 20 and 30 nucleotides in length, between 21 and 29 nucleotides in length or between about 20 and 28 nucleotides in length.

The present nucleic acid amplification product to be analyzed may stem from the amplification of genomic DNA, mitochondrial DNA, chloroplast DNA, cDNA, or the like, from any suitable source. The fragments (target) may have any suitable length, such as about 10 to 10,000, or 20 to 2,000 nucleotides, among others. The fragments may or may not be size-selected before attachment to the adapters (primer regions). Fragments may be generated from a source nucleic acid material by any suitable approach, such as shearing, chemical digestion, enzymatic digestion, amplification with one or more primers, reverse transcription, end-polishing, or any combination thereof, among others. The fragments may have flush or overhanging ends and may be at least predominantly double-stranded or single-stranded. The target nucleic acid may be RNA or DNA. DNA is preferred.

In a preferred embodiment the upstream and downstream primer region stems from the amplification of the library adaptors. So, a library is generated by ligating adaptors to the inserts which then in turn serve as upstream and downstream primer regions. Each adapter (or adapter region) may have any suitable structure before and/or after attachment to inserts. The adapter before attachment may include a nucleic acid or nucleic acid analog. Each adapter may be formed by one or more oligonucleotide strands each having any suitable length, such as at least about 6, 8, 10, 15, 20, 30, or 40 nucleotides, among others, and/or less than about 200, 100, 75, or 50 nucleotides, among others. The adapter may be provided by one or more oligonucleotides that are chemically synthesized in vitro. The adapter may be configured to be attached to inserts at only one of its two ends. In some cases, the adapter may be partially or completely single-stranded before attachment to inserts, such as if the adapter is provided by a primer that attaches to inserts via primer extension. The adapter of the library principally serves as upstream and downstream primer region. The target region of the present invention is herein preferably the amplified library insert.

Ideally in the method according to the invention the primers are between 10 nucleotides and 22 nucleotides in length and/or the probes are between 8 and 17 nucleotides in length.

Preferably the primers are between 11 and 17 nucleotides in length, more preferably they are between 12 and 16 nucleotides in length. They need not have the same length and may differ.

Preferably the probes are between 9 and 16 nucleotides in length, more preferably they are between 11 and 14 nucleotides in length. They need not have the same length and may differ.

Preferably, the combined length of primer+probe is between 18 nucleotides and 35 nucleotides. More preferably the combined length is between 23 nucleotides and 30 nucleotides in length.

Preferably in the method of the invention there are two primers, a first upstream and a second downstream primer. A first probe lies adjacent to and downstream of said first upstream primer. The first primer and first probe may have the same 5′-3′ orientation or in alternative embodiment the first probe binds the opposite strand and has an 3′-5′ orientation whereas, the first primer of course is oriented in a 5′-3′ manner.

A second probe will lie adjacent to and downstream of the second so called downstream primer. Also here, this probe may bind the same strand and have the same 3′-5′ orientation or may bind the opposite strand.

Preferably in the method of the invention two duplex scorpions are used as probes.

The four alternative embodiments are shown in FIG. 2. All four alternatives are feasible. In variants (A) and (B) the two probes specifically bind to opposite strands of the library fragment. In variant (A) both probes bind in close proximity downstream of each primer. Due to the short distance between both primers and probes, variant (A) has the advantage of a very efficient release of fluorescence signal by the 5′-3′ exonuclease activity of Taq polymerase during strand polymerization. In variant (B) both probes bind to the distant second adapter sequences downstream of the primer binding sites. Variant (B) has the advantage, that each fluorescence signal depicts the polymerization of a fragment that spans the library fragment from one adapter to the other. A disadvantage of variant (B) is that for longer fragments (>500 bp) amplification efficiency goes down leading to less release of fluorescence signal. In variants (C) and (D) the two probes specifically bind to the same strand of the library fragment. In both variants one probe binds in close proximity downstream of one primer and the second probe binds to the second adapter sequence downstream of the same primer. This has the advantage that a single elongation step creates a double signal which depicts the polymerization of a fragment that spans the library fragment from one adapter to the other.

In the inventive method preferably at least one of the primers comprises one or more locked nucleic acid nucleotides (LNA) or another nucleotide analogue that increase template binding strength. LNA was the first nucleotide analogue synthesized with its sugar locked into the C3′-endo conformation. This analogue exhibits significantly increased target-binding affinity (ΔTm/modification=approximately +5° C. compared with natural DNA). Other modified nucleotides are available; See Chem Commun (Camb). 2017 Aug. 14; 53(63): 8910-8913, Published online 2017 Jul. 27. doi: 10.1039/c7cc05159j, PMCID: PMC5708354, PMID: 28748236.

Locked nucleic acid (LNA) enhances binding affinity of triazole-linked DNA towards RNA†

In the inventive method preferably at least one of the probes comprises one or more locked nucleic acid nucleotides (LNA) or another nucleotide analogue that increase template binding strength.

In a preferred embodiment all primers and probes comprise LNAs. The skilled person will be able to determine the ideal amount of LNA nucleotides per probe or primer.

In a preferred embodiment the one or more primers comprise between 1 and 8 LNA nucleotides, 2 and 7 LNA nucleotides, 3 and 6 LNA nucleotides or 4 and 5 LNA nucleotides. The amount will depend also on the length of the primer.

Ideally the one or more probes comprise between 2 and 12 LNA nucleotides, 3 and 11 LNA nucleotides, 4 and 10 LNA nucleotides, 5 and 9 LNA nucleotides, 6 and 8 LNA nucleotides or about 7 LNA nucleotides. The amount will depend also on the length of the probe.

In a preferred embodiment the amplification product from step a) stems from amplifying a nucleic acid library. In a further preferred embodiment, the library is a sequencing library.

Thermophiles, like other bacteria, contain five types of DNA polymerases, termed polymerase I, II, III, IV, and V. Given the nature of thermophile habitats, these enzymes typically exhibit thermostability, and are generally referred to as thermostable DNA polymerases. DNA polymerase I (“Pol I”) is the most abundant polymerase and is generally responsible for certain types of DNA repair, including a repair-like reaction that permits the joining of Okazaki fragments during DNA replication. Pol I is essential for the repair of DNA damage induced by UV irradiation and radiomimetic drugs. DNA polymerase II is thought to play a role in repairing DNA damage that induces the SOS response. In mutants that lack both Pol I and DNA polymerase III, DNA polymerase II repairs UV-induced lesions. DNA polymerase II is a multi-subunit replicase.

Thermostable DNA polymerases have proven very useful in several applications in molecular biology. One such application is the polymerase chain reaction (PCR). The PCR process is described, for example, in U.S. Pat. Nos. 4,683,195 and 4,683,202, the disclosures of which are incorporated herein by reference. In a PCR reaction, primers, template, and nucleoside triphosphates are combined in appropriate buffer with a DNA polymerase, for the basic steps of thermal denaturation of target DNA, hybridization of primers to template with cooling of the reaction mixture, and primer extension to produce extension products complementary to template sequences. Thermal denaturation is repeated, primers are annealed to extension products with cooling of the reaction mixture, and previously produced extension products serve as templates for subsequent primer extension reactions. This cycle is repeated a number of times, resulting in an exponential amplification of the desired nucleic acid sequence. Use of a thermostable DNA polymerase provides for repeated heating/cooling cycles without loss of enzyme activity. Preferred polymerases herein are, Taq polymerase, Vent polymerase, Deep Vent polymerase, Bst polymerase, Pfu polymerase, Tth polymerase and the like. Ideally and preferably the polymerase has displacement activity and/or an 5′-3′ exonuclease activity and will displace the probe and free the label. The probe is preferably a TaqMan probe comprising a label and a quencher.

The invention relates also to the following nucleic acids which are explicitly claimed herein (see SEQ ID NO.: 1-24). The kit of the invention may use the following primers and probes claimed herein.

In a preferred embodiment the primers and probes bind in the p5 and p7 regions of the Illumina library adaptors; See FIG. 4.

| Illumina P5 | |
|---|---|
| sequence | NNNNNN-Index sequences (variable sequence) |
| 5′-3′ | 5′-AAT GAT ACG GCG ACC ACC GAG ATC TAC AC-3′ |
| sequence | |
| (SEQ ID NO. | |
| 1) (29 | |
| nucleotides) | |
| Opposite | 5′-GTG TAG ATC TCG GTG GTC GCC GTA TCA |
| strand in | TT-3′ |
| 5′-3′ | |
| orientation | |
| (SEQ ID NO. | |
| 2) | |
| (29 | |
| nucleotides) | |
| Illumina P7 | |
| sequence | |
| 5′-3′ | 5′-ATC TCG TAT GCC GTC TTC TGC TTG-3′ |
| sequence | |
| (SEQ ID NO. | |
| 3) | |
| (24 | |
| nucleotides) | |
| Opposite | 5′-CAA GCA GAA GAC GGC ATA CGA GAT-3′ |
| strand in | |
| 5′-3′ | |
| orientation | |
| (SEQ ID NO. | |
| 4) | |
| (24 | |
| nucleotides) | |
| Truseq | 5′- |
| Single Index | AATGATACGGCGACCACCGAGATCTACACTCTTTCCCTACACGACGCTCT |
| Library (SEQ | TCCGATCT-insert- |
| ID NO 5) | AGATCGGAAGAGCACACGTCTGAACTCCAGTCACNNNNNNNNATCTCGTA |
| | TGCCGTCTTCTGCTTG-3′ |
| Truseq | 5′- |
| Single Index | CAAGCAGAAGACGGCATACGAGATNNNNNNNNGTGACTGGAGTTCAGACG |
| Library (SEQ | TGTGCTCTTCCGATCT-insert- |
| ID NO 6) | AGATCGGAAGAGCGTCGTGTAGGGAAAGAGTGTAGATCTCGGTGGTCGCC |
| | GTATCATT-3′ |
| Truseq Dual | 5′- |
| Index | AATGATACGGCGACCACCGAGATCTACACNNNNNNNNACACTCTTTCCCT |
| Library A: | ACACGACGCTCTTCCGATCT-insert- |
| (SEQ ID NO | AGATCGGAAGAGCACACGTCTGAACTCCAGTCACNNNNNNNNATCTCGTA |
| 7) | TGCCGTCTTCTGCTTG-3′ |
| Truseq Dual | 5′- |
| Index | CAAGCAGAAGACGGCATACGAGATNNNNNNNNGTGACTGGAGTTCAGACG |
| Library B: | TGTGCTCTTCCGATCT-insert- |
| (SEQ ID NO | AGATCGGAAGAGCGTCGTGTAGGGAAAGAGTGTNNNNNNNNGTGTAGATC |
| 8) | TCGGTGGTCGCCGTATCATT-3′ |
| Nextera Dual | 5′- |
| Index | AATGATACGGCGACCACCGAGATCTACACNNNNNNNNTCGTCGGCAGCGT |
| Library A: | CAGATGTGTATAAGAGACAG-insert- |
| (SEQ ID NO | CTGTCTCTTATACACATCTCCGAGCCCACGAGACNNNNNNNNATCTCGTA |
| 9) | TGCCGTCTTCTGCTTG-3′ |
| Nextera Dual | 5′- |
| Index | CAAGCAGAAGACGGCATACGAGATNNNNNNNNGTCTCGTGGGCTCGGAGA |
| Library B: | TGTGTATAAGAGACAG-insert- |
| (SEQ ID NO | CTGTCTCTTATACACATCTGACGCTGCCGACGANNNNNNNNGTGTAGATC |
| 10) | TCGGTGGTCGCCGTATCATT-3′ |
| NEBnext | 5′- |
| Library A: | AATGATACGGCGACCACCGAGATCTACACTCTTTCCCTACACGACGCTCT |
| (SEQ ID NO | TCCGATCT-insert- |
| 11) | AGATCGGAAGAGCACACGTCTGAACTCCAGTCACNNNNNNATCTCGTATG |
| | CCGTCTTCTGCTTG-3′ |
| NEBnext | 5′- |
| Library B: | CAAGCAGAAGACGGCATACGAGATNNNNNNGTGACTGGAGTTCAGACGTG |
| (SEQ ID NO | TGCTCTTCCGATCT-insert- |
| 12) | AGATCGGAAGAGCGTCGTGTAGGGAAAGAGTGTAGATCTCGGTGGTCGCC |
| | GTATCATT-3′ |
| GeneRead | 5′- |
| Library Prep | AATGATACGGCGACCACCGAGATCTACACNNNNNNNNACACTCTTTCCCT |
| Kit I A: | ACACGACGCTCTTCCGATCT-insert- |
| (SEQ ID NO | CAAGCAGAAGACGGCATACGAGATNNNNNNNNATCTCGTATGCCGTCTTC |
| 13) | TGCTTG-3′ |
| GeneRead | 5′- |
| Library Prep | CAAGCAGAAGACGGCATACGAGATNNNNNNNNATCTCGTATGCCGTCTTC |
| Kit I B: | TGCTTG-insert- |
| (SEQ ID NO | AGATCGGAAGAGCGTCGTGTAGGGAAAGAGTGTNNNNNNNNGTGTAGATC |
| 14) | TCGGTGGTCGCCGTATCATT-3′ |
| QIAseq | 5′- |
| stranded | AATGATACGGCGACCACCGAGATCTACACGTTCAGAGTTCTACAGTCCGA |
| mRNA Library | CGATC-insert- |
| Prep Kit: | AACTGTAGGCACCATCAATNNNNNNNNNNNNAGATCGGAAGAGCACACGT |
| (SEQ ID NO | CTGAACTCCAGTCACACAAGACGATCTCGTATGCCGTCTTCTGCTTG-3′ |
| 15) | |
| FIG. 4: | Examples for primer and probe designs (+ indicates |
| | the nucleotide after the + is an LNA) and |
| | exemplary ILLUMINA library adapters : |
| Forward | |
| primers: | |
| ILMN quant | 5′-A+ATGATA+CGGC+GACC-3′ |
| F4 (SEQ | |
| ID NO 16) | |
| ILMN quant | 5′-ATGATACGGCGA+CCAC-3′ |
| F5 (SEQ ID | |
| NO 17) | |
| ILMN quant | 5′-AA+TGA+TAC+GGCG+AC-3′ |
| F6 (SEQ ID | |
| NO 18) | |
| Reverse | |
| primer: | |
| ILMN quant | 5′-CA+AGCA+GAAGAC+GG-3′ |
| R5 (SEQ ID NO | |
| 19) | |
| FAM labelled | |
| probe: | |
| IL_LQ_P06_FAM | 5′-C+G+A+G+AT+CT+A+CAC-3′ |
| (SEQ ID NO | |
| 20) | |
| HEX-labelled | |
| probes: | |
| IL_LQ_P08_HEX | 5′-AT+CT+C+G+TA+T+GCC-3′ |
| (SEQ ID NO | |
| 21) | |
| IL_LQ_P09_HEX | 5′-A+T+CTC+G+TA+TG+CC-3′ |
| (SEQ ID NO | |
| 22) | |
| IL_LQ_P10_HEX | 5′-T+C+T+C+GTA+TG+CC-3′ |
| (SEQ ID NO | |
| 23) | |
| ILLUMINA | 5′- |
| library | AATGATACGGCGACCACCGAGATCTACAC[i]ACACTCTTTCCCTACACG |
| fragment | ACGCTCTTCCGATCGATCGGAAGAGCACACGTCTGAACTCCAGTCAC[i] |
| with double | ATCTCGTATGCCGTCTTCTGCTTG-3′ |
| indexed | |
| adapter | |
| sequences (SEQ | |
| ID NO 24) | |

The invention also relates to a nucleic acid selected from the group of:

| FIG. 4: | Examples for primer and probe designs (+ indicates |
|---|---|
| | the nucleotide after the + is an LNA) and |
| | exemplary ILLUMINA library adapters: |
| Forward | |
| primers: | |
| ILMN quant | 5′-A+ATGATA+CGGC+GACC-3′ |
| F4 (SEQ | |
| ID NO 16) | |
| ILMN quant | 5′-ATGATACGGCGA+CCAC-3′ |
| F5 (SEQ ID | |
| NO 17) | |
| ILMN quant | 5′-AA+TGA+TAC+GGCG+AC-3′ |
| F6 (SEQ ID | |
| NO 18) | |
| Reverse | |
| primer: | |
| ILMN quant | 5′-CA+AGCA+GAAGAC+GG-3′ |
| R5 (SEQ ID NO | |
| 19) | |
| FAM labelled | |
| probe: | |
| IL_LQ_P06_FAM | 5′-C+G+A+G+AT+CT+A+CAC-3′ |
| (SEQ ID NO | |
| 20) | |
| HEX-labelled | |
| probes: | |
| IL_LQ_P08_HEX | 5′-AT+CT+C+G+TA+T+GCC-3′ |
| (SEQ ID NO | |
| 21) | |
| IL_LQ_P09_HEX | 5′-A+T+CTC+G+TA+TG+CC-3′ |
| (SEQ ID NO | |
| 22) | |
| IL_LQ_P10_HEX | 5′-T+C+T+C+GTA+TG+CC-3′ |
| (SEQ ID NO | |
| 23) | |

wherein ‘+’ indicates the nucleotide after the ‘+’ is a LNA (locked nucleic acid).

Method according to the present invention wherein the primers and/or probes are located in the underlined regions:

| | |
|---|---|
| Truseq | 5′- |
| Single Index | AATGATACGGCGACCACCGAGATCTACACTCTTTCCCTACACGACGCTCT |
| Library (SEQ | TCCGATCT-insert- |
| ID NO 5) | AGATCGGAAGAGCACACGTCTGAACTCCAGTCACNNNNNNNNATCTCGTA |
| | TGCCGTCTTCTGCTTG-3′ |
| Truseq | 5′- |
| Single Index | CAAGCAGAAGACGGCATACGAGATNNNNNNNNGTGACTGGAGTTCAGACG |
| Library (SEQ | TGTGCTCTTCCGATCT-insert- |
| ID NO 6) | AGATCGGAAGAGCGTCGTGTAGGGAAAGAGTGTAGATCTCGGTGGTCGCC |
| | GTATCATT-3′ |
| Truseq Dual | 5′- |
| Index | AATGATACGGCGACCACCGAGATCTACACNNNNNNNNACACTCTTTCCCT |
| Library A: | ACACGACGCTCTTCCGATCT-insert- |
| (SEQ ID NO | AGATCGGAAGAGCACACGTCTGAACTCCAGTCACNNNNNNNNATCTCGTA |
| 7) | TGCCGTCTTCTGCTTG-3′ |
| Truseq Dual | 5′- |
| Index | CAAGCAGAAGACGGCATACGAGATNNNNNNNNGTGACTGGAGTTCAGACG |
| Library B: | TGTGCTCTTCCGATCT-insert- |
| (SEQ ID NO | AGATCGGAAGAGCGTCGTGTAGGGAAAGAGTGTNNNNNNNNGTGTAGATC |
| 8) | TCGGTGGTCGCCGTATCATT-3′ |
| Nextera Dual | 5′- |
| Index | AATGATACGGCGACCACCGAGATCTACACNNNNNNNNTCGTCGGCAGCGT |
| Library A: | CAGATGTGTATAAGAGACAG-insert- |
| (SEQ ID NO | CTGTCTCTTATACACATCTCCGAGCCCACGAGACNNNNNNNNATCTCGTA |
| 9) | TGCCGTCTTCTGCTTG-3′ |
| Nextera Dual | 5′- |
| Index | CAAGCAGAAGACGGCATACGAGATNNNNNNNNGTCTCGTGGGCTCGGAGA |
| Library B: | TGTGTATAAGAGACAG-insert- |
| (SEQ ID NO | CTGTCTCTTATACACATCTGACGCTGCCGACGANNNNNNNNGTGTAGATC |
| 10) | TCGGTGGTCGCCGTATCATT-3′ |
| NEBnext | 5′- |
| Library A: | AATGATACGGCGACCACCGAGATCTACACTCTTTCCCTACACGACGCTCT |
| (SEQ ID NO | TCCGATCT-insert- |
| 11) | AGATCGGAAGAGCACACGTCTGAACTCCAGTCACNNNNNNATCTCGTATG |
| | CCGTCTTCTGCTTG-3′ |
| NEBnext | 5′- |
| Library B: | CAAGCAGAAGACGGCATACGAGATNNNNNNGTGACTGGAGTTCAGACGTG |
| (SEQ ID NO | TGCTCTTCCGATCT-insert- |
| 12) | AGATCGGAAGAGCGTCGTGTAGGGAAAGAGTGTAGATCTCGGTGGTCGCC |
| | GTATCATT-3′ |
| GeneRead | 5′- |
| Library Prep | AATGATACGGCGACCACCGAGATCTACACNNNNNNNNACACTCTTTCCCT |
| Kit I A: | ACACGACGCTCTTCCGATCT-insert- |
| (SEQ ID NO | CAAGCAGAAGACGGCATACGAGATNNNNNNNNATCTCGTATGCCGTCTTC |
| 13) | TGCTTG-3′ |
| GeneRead | 5′- |
| Library Prep | CAAGCAGAAGACGGCATACGAGATNNNNNNNNATCTCGTATGCCGTCTTC |
| Kit I B: | TGCTTG-insert- |
| (SEQ ID NO | AGATCGGAAGAGCGTCGTGTAGGGAAAGAGTGTNNNNNNNNGTGTAGATC |
| 14) | TCGGTGGTCGCCGTATCATT-3′ |

The invention also relates to a nucleic acid amplification composition comprising:
a. at least two primers having a length of between 10 and 22 nucleotides and optionally comprising one or more locked nucleic acid nucleotide analogues or another nucleotide analogue that increase template binding strength and,
b. at least two oligonucleotide probes having a length of between 8 and 17 nucleotides and optionally comprising one or more locked nucleic acid nucleotide analogues or another nucleotide analogue that increase template binding strength.

The invention also relates to the use of a composition according to the invention for the analysis of a nucleic acid library.

Preferably the library has adaptors and said adaptors have a conserved region for amplification and preferably said target region is between 15 and 35 nucleotides in length, between 16 and 34 nucleotides in length, between 17 and 33 nucleotides in length, between 18 and 32 nucleotides in length, between 19 and 31 nucleotides in length, between 20 and 30 nucleotides in length, between 21 and 29 nucleotides in length or between about 20 and 28 nucleotides in length.

The invention also relates to a kit comprising:
a. at least two primers have a length of between 10 and 22 nucleotides and optionally comprising one or more locked nucleic acid nucleotide analogues or another nucleotide analogue that increase template binding strength and,
b. at least two oligonucleotide probes having a length of between 8 and 17 nucleotides and optionally comprising one or more locked nucleic acid nucleotide analogues or another nucleotide analogue that increase template binding strength.

Preferably, the method, composition, or kit according to the invention has probes that are hydrolysis probes, and the oligonucleotide probes carry a label and, wherein the label on the two probes differs.

Preferably, the method, composition, or kit according to the invention has a nucleic acid amplification product that stems from amplifying a nucleic acid library, wherein the library is a sequencing library. Preferably the sequencing library is an ILLUMINA library.

Sequencing libraries are created to perform diverse analyses, such as whole genome sequencing, whole exome sequencing, targeted DNA sequencing, whole-transcriptome sequencing, targeted RNA sequencing, ChIP-seq, RIP-seq, epigenetic studies and more. An ILLUMINA library is intended to be run on an ILLUMINA sequencing platform, e.g. the MiSeq system, the NextSeq systems or NovaSeq system. All ILLUMINA library types use two types of sequencing adapters that each shares one of the two adapter sequences P5 and P7 at its distal ends.

Depending on the intended analysis different library preparation kits are used. This results in ILLUMINA libraries that all share the P5 and P7 adapter regions but can substantially differ in the composition of the rest of the adapter sequences. FIG. 1 illustrates examples of ILLUMINA library preparation kits with differing adapter sequences.

Preferably in the method according to the invention the amplification step b) is a digital PCR assay and the template is subdivided into partitions or droplets.

### EXAMPLES

### Example 1

### QIAcuity System and Workflow

The QIAcuity is designed as a walk-away instrument that integrates and automates all plate processing steps. Only the plate preparation must be done manually before starting the run. This includes the pipetting of the target, reagents, and master mix in the plate's input wells and the closing of the wells with the nanoplate seal. Once the preparation is done and the experiment is set up, the plate is placed in a free plate slot of the instrument tray. By reading the barcode of the plate, the instrument links the plate to the experiment previously defined in the software. After pressing the play button, all further steps are performed fully automated by the instrument.

### Partitioning

In the first step, the plate's microchannels and partitions are filled with the input volume in the wells. This is done by plunging of 24/96 pins in the elastic top seal and the input wells. This creates a peristaltic pressure that pumps the input well liquid into the microchannels and partitions.

Subsequentially, the connecting channels between the partitions are being closed by a pressure controlled rolling process; See FIG. 8.

### Thermocycling

The second step is a high-accuracy plate thermocycler that performs the polymerase chain reaction. The cycling profile can be set in the QIAcuity Software Suite or the instrument software. The thermal cycler of the QIAcuity is a plate thermocycler with high speed and precise temperature control of the various cycling steps. Several Peltier elements are used for the temperature generation and control. For optimal thermal contact between plate and thermocycler, the plate is being clamped on the heating surface during cycling.

### Imaging

The final step is the image acquisition of all wells. The user can select the detection channels in the experiment setup. The partitions that have a target molecule inside emit fluorescence light and are brighter than those without target.

### Calculation of the Concentration of Target Library Fragments in the Analysed Sample

After endpoint PCR individual wells of the nanoplate are imaged in both channels green and yellow by the imaging module of the dPCR instrument. Images are analysed by the QIAcuity Software Suite. In this analysis, each partition of the well is defined as valid or non-valid based on distinct signal criteria. For each partition in the well a relative fluorescence value is calculated for the yellow and the green channel. To distinguish positive from a negative signals the software suite sets an automatic threshold in both channels, green and yellow. This results in distinct signal populations of partitions that are double negative (0), only show green (G) or yellow (Y) signal and partitions that are positive for both channels (GY) (illustrated in the two-dimensional scatterplot in FIG. 9). The QIAcuity Software suite provides export tables that list for each well the total numbers of these 4 types of partitions, the cycled volume and the total number of valid partitions.

Using these numbers the concentration of target fragments in the library can be calculated using Poisson statistics. Statistical calculation were taken from Regan J F, Kamitaki N, Legler T, Cooper S, Klitgord N, et al. (2015) A Rapid Molecular Approach for Chromosomal Phasing.

Therein, the linkage of both signals on one target fragment is considered for the calculation. A target fragment is a DNA fragment that is linked to both adapters and produces two signals, green and yellow (GY). As Illumina NGS libraries may also contain non-target fragments that produce only one of the two signals, partitions with only green or yellow signal are expected. This can lead to partitions with a double positive signal that originates from a by-chance co-localization of single signal fragments in one partition. The number of these by-chance double positive partitions (Nch) is calculated by:

Nch=NG*NY/N0 Equation 1:

Where N denotes the number of partitions, so that NY and NG are the counts of single positive partitions, N0 are the counts of double negative partitions showing no fluorescence signal and Nch the by-chance double positive partitions showing both fluorescence signals.

In the presence of target fragments with linked fluorophore signals (GY) and fragments with single signals (G and Y), there will be additional double positive partitions. Double positive signals can originate from 5 different combinations of fragments in a single partition. That is G+Y, G+GY, Y+GY, GY and GY+G+Y. The combination of G+Y has been considered in Equation 1. For the calculation of the total number of GY a combination with G and Y in the partition can be neglected as in all of these cases a GY is present.

The total number of partitions that do not contain target fragments with linked fluorophore signals is calculated using:

NnotGY=N0+NG+NY+Nch Equation 2:

From this the concentration A (average number of copies/partition) of target fragments with linked fluorophores is calculated using:

λGY=ln(Ntot)−ln(NnotGY) Equation 3:

Where Ntot is the total number of valid partitions.

The concentration (in copies/μl) of target fragments with linked fluorophore signal in the reaction is calculated using:

c(GY)=λGY*Ntot/cycled volume Equation 4:

Example of an output table of multi occupancy counts for a single well is shown below. The table summarizes for well E5 total numbers (Count) of the 4 different partitions based on their assigned fluorescence signatures (Group), the total number of valid partitions (Total) and the summed cycled volume of all valid partitions in μl (Volume). The partition groups ++ (double positive), +− (positive in first category), −+ (positive in second category) and −− (double negative) correspond to the Signal order given in the Categories column.

| Well | Hyperwell | Categories | Group | Count | Total | Volume |
|---|---|---|---|---|---|---|
| E5 | − | GREEN-YELLOW | ++ | 1529 | 8255 | 2.889 |
| E5 | − | GREEN-YELLOW | +− | 150 | 8255 | 2.889 |
| E5 | − | GREEN-YELLOW | −+ | 111 | 8255 | 2.889 |
| E5 | − | GREEN-YELLOW | −− | 6465 | 8255 | 2.889 |

### Example 2

During NGS library preparation target DNAs are linked to DNA adapters. Each target DNA is linked to two different adapters forming a library fragment ready for sequencing. A precise quantification of these full-length library fragments in NGS libraries is an important step in the QC of next generation sequencing. The present invention allows quantification of these fragments in Illumina NGS libraries using digital PCR. Both adapters of Illumina NGS libraries have a conserved and a variable region. The variable region differs in length and sequence composition between different library types whereas the conserved regions P5 and P7 are identical for all Illumina library types (see figure below). The assay for captures all Illumina library types with a single assay. Therefore, the design of both duplex scorpions specifically target these two regions. This requires the design of short oligos as P5 is only 29 bp and P7 is only 24 bp in length (see figure below). To adapt the oligos to the conditions required for successful PCR and binding of the scorpion fluorophore sequence to the extended scorpion primer sequence the assay designs use locked nucleic acids (LNAs). A detailed summary of the sequence composition of the oligos is given in the materials and methods section below.

The assay of the product consists of two duplex scorpions that each target the opposite strand at one of the two conserved adapter sequences as indicated in FIG. 10. Each Duplex Scorpion consists of two oligos, a fluorophore primer and a quencher oligo that can form a duplex. The fluorophore primer consists of three parts, the fluorophore domain, the HEG (hexethylene glycol) spacer and the annealing primer domain. The fluorophore domain contains a fluorophore at the 5′end and a sequence that is reverse-complementary to the extension sequence down-stream of the binding site of the primer domain. The HEG spacer enables flexibility for the fluorophore domain to flip and bind to the extended primer during amplification. The HEG further prevents extension by the polymerase. The primer domain consists of a primer sequence that specifically bind to the conserved adapter sequence. The quencher oligo contains a quencher at the 3′end and specifically hybridizes with the fluorophore domain of the fluorophore primer. There is one possible design variant with 2 duplex scorpions each specific to one of the two adapters. Oligo parameters are listed in the table below.

Summary of assay design framework

| | | Number | Modifications | | |
|---|---|---|---|---|---|
| Oligo | Length | of LNAs | 5′ | internal | 3′ |
| 1st Fluorophore | 25-33 | 4-10 | FAM | HEG | none |
| primer | | | | spacer | |
| 2nd Fluorophore | 25-33 | 4-10 | HEX | HEG | none |
| primer | | | | spacer | |
| 1st Quencher oligo | 9-15 | 1-5 | none | none | IBFQ or BHQ1 |
| 2nd Quencher | 9-15 | 1-5 | none | none | IBFQ or BHQ1 |
| oligo | | | | | |

To create a signal for complete fragments both Scorpion primers have to bind to the target sequences on opposite strands. Illumina NGS library fragments with both adapters will bind both scorpion primers and release two fluorescent signals.

At the beginning of the PCR fluorophore primer and quencher oligo are in duplex (FIG. 11). In this bound form the fluorophore of the fluorophore primer and the quencher of the quencher oligo are in close proximity and fluorescence is quenched, so no signal can be detected. In the presence of a NGS library fragment with both adapters both fluorophore primers will bind their specific target sequences. The primer will be extended. During denaturation the quencher oligo is separated from the fluorophore primer releasing the fluorescence signal of the fluorophore. When cooling down for annealing the fluorophore domain of the fluorophore primers that have been extended bind/hybridize intramolecularly to the sequence that is downstream of the primer. Back hybridization events to quencher oligos are very rare as the intramolecular binding is kinetically much more favorable. Only the non-extended fluorophore primers bind again to a quencher and their fluorescence is again quenched. Extension of the reverse primer will lead to hydrolysis of the fluorophore of the intramolecularly bound fluorophore domain due to the exonuclease activity of the polymerase. With each cycle more and more intramolecular bound fluorophore domains are produced creating a strong fluorescence signal at the end of the PCR reaction.

This creates a double fluorescence signal in each partition that contains NGS fragments with both adapters, P5 and P7. In digital PCR the fragments are randomly distributed into thousands of partitions in which endpoint PCR creates binary values for the presence and absence of the template of interest for each partition. After correction for the possibility of having one or more template molecules per partition using poisson statistics, these binary values reveal absolute quantification of molecules/μl. For the assay design of the product the binary signal is based on the presence and absence of a double positive signal for both fluorophores of the two probes.

Our invention allows quantification of Illumina NGS library fragments using digital PCR for example on the QIAcuity nanoplate dPCR system. During endpoint PCR in the partitions of the dPCR reaction the amplification of each complete NGS library fragment produces two fluorescent signals each specific for one of the two conserved adapter sequences P5 an P7. For example, the QIAcuity software automatically detects and quantifies partitions with a double positive fluorescent signal and calculates, based on Poisson statistics, the absolute number of double positive library fragments in the template and the corresponding concentration in the library.

In a preferred embodiment of the invention, we provide a kit that contains one assay in one tube plus two tubes of 1 ml H₂O and one tube of 1 ml QIAcuity Probe mastermix. The assay consists of 2 duplex scorpion primers (2 fluorophore primers and 2 quencher oligos), premixed in one tube.

dPCR Setup

In the first step, setting up the dPCR reaction, two dilutions of the Illumina libraries to be quantified are prepared. The degree of dilution depends on the expected concentration of the respective library. For the dPCR reaction, a mix of defined amounts of assay components (primer and probe mix), master mix and water is placed in a reaction tube. A defined amount of pre-diluted library is added to the mix and mixed well. The final mix is transferred to the well of a dPCR 8.5K nanoplate, which is then sealed and loaded into the QIAcuity dPCR instrument.

dPCR Reaction

To start the dPCR run, the appropriate protocols for partitioning, cycling and imaging must be specified by the user in the QIAcuity software suite. The assay requires specific cycling and imaging conditions according to the assay designs and fluorophores of the probes used. Protocols for the dPCR reaction are provided together with the developed kit.

### Data Analysis

After the imaging step, the acquired data are analyzed using the QIAcuity software suite. The assay product is designed in a way that the endpoint PCR of the dPCR reaction reveals signal intensities of positive partitions that are strong enough for the software to distinguish positive from negative partitions. The software's auto-threshold function performs this discrimination in both signal channels. This indicates for each partition in the well whether no, one or both signals have been detected. Partitions with a positive signal for both channels, so-called multi-occupancy partitions, represent the target partitions. The number of multi-occupancy partitions, valid partitions, cycled volume and other parameters of each well are summarized in a table that has to be exported from the Software suite for further analyses. Using an external excel data sheet the concentration of NGS library fragments with double positive signals is calculated from the data in the exported table. Calculations apply Poisson statistics.

### Data Output and Interpretation

The duplex scorpion assay design applied to NGS library fragments in digital PCR on the QIAcuity will create data that enables the same readout and data interpretation as with the previous design based on two probes and two primers. We currently test if we do see the same difference in relative fluorescence units (RFU) signal intensity for short and long NGS library fragments. First test results indicate a similar negative correlation of RFU signal intensity and library fragment length (FIG. 13).

### Materials and Methods

### Protocols and Procedures

A single reaction in the 96-well 8.5K nanoplate contains the following reagents in the given concentrations:

| Reagent | 1x | final |
|---|---|---|
| QIAcuity MM 4x | 3.00 | 1x |
| PriProMix (10 μM) | 0.48 | 0.40 μM |
| PriProMix (10 μM) | 0.48 | 0.40 μM |
| Quencher (10 μM) | 1.44 | 1.20 μM |
| Quencher (10 μM) | 1.44 | 1.20 μM |
| Water | 1.16 | |
| Template | 4 | |

12 μl

The dPCR reaction is run on the QIAcuity dPCR system using the following cycling and system settings:

| Temperature | Duration in min | Cycles |
|---|---|---|
| 93° C. | 02:00 | 1x |
| 93° C. | 00:15 | 40x |
| 58° C. | 01:15 | |
| 45° C. | 05:00 | 1x |

Imaging settings

| Channel | Exposure time (ms) | Gain |
|---|---|---|
| GREEN | 500 | 6 |
| YELLOW | 500 | 6 |

Priming Profile

| | |
|---|---|
| PrimeTravelDistance | 2, 6 |
| Velocity (mm/sec) | 4 |

### Examples of Duplex Scorpion Designs

The invention relates also to the following nucleic acids which are explicitly claimed herein (see SEQ ID NO. 25-44). Various designs were tested. Nucleotides after + are LNAs. HEG is a hexethylene glycol. FAM and HEX are dyes. Q indicates the Quencher. In accordance with WIPO Standard 26, sequences which are separated by a HEG spacer are assigned two SEQ ID NOs representing each part separately, the sequence of the second SEQ ID NO is underlined.

| Name | Sequence | length | |
|---|---|---|---|
| P06-F5 | 5′-FAM-GT+G+TA+GA+T+C+T+CG- | (length 28) | SEQ ID NO. 25 and |
| | HEG-ATGATACGGCGA+CCAC-3′ | | SEQ ID NO. 26 |
| P06-ASQ | 5′-CGAGATCTACAC-Q-3′ | (length 12) | SEQ ID NO. 27 |
| P06v4-F5 | 5′-FAM-T+G+TA+GA+T+C+T+CG- | (length 27) | SEQ ID NO. 28 and |
| | HEG-ATGATACGGCGA+CCAC-3′ | | SEQ ID NO. 29 |
| P06v4- | 5′-CGAGATCTACA-Q-3′ | (length 11) | SEQ ID NO. 30 |
| ASQ | | | |
| P09-R5 | 5′-HEX-A+T+CTC+G+TA+TG+CC- | (length 26) | SEQ ID NO. 31 and |
| | HEG-CA+AGCA+GAAGAC+GG-3′ | | SEQ ID NO. 32 |
| P15-R5 | 5′-HEX-A+T+CT+C+GT+AT+GCC- | (length 26) | SEQ ID NO. 33 and |
| | HEG-CA+AGCA+GAAGAC+GG-3′ | | SEQ ID NO. 34 |
| P09/15- | 5′-GGCATACGAGAT-Q-3′ | (length 12) | SEQ ID NO. 35 |
| ASQ | | | |
| P06-F8 | 5′-FAM-GT+G+TA+GA+T+C+T+CG- | (length 30) | SEQ ID NO. 36 and |
| | HEG-AGCTAATGATACGGCGAC-3′ | | SEQ ID NO. 37 |
| P06-ASQ | 5′-CGAGATCTACAC-Q-3′ | (12 length) | SEQ ID NO. 38 |
| P06v4-F8 | 5′-FAM-T+G+TA+GA+T+C+T+CG- | (length 29) | SEQ ID NO. 39 and |
| | HEG-AGCTAATGATACGGCGAC-3′ | | SEQ ID NO. 40 |
| P06v4- | 5′-CGAGATCTACA-Q-3′ | (length 11) | SEQ ID NO. 41 |
| ASQ | | | |
| P15-R8 | 5′-HEX-A+T+CT+C+GT+AT+GCC- | (length 28) | SEQ ID NO. 42 and |
| | HEG-AGCTCA+AGCA+GAAGAC | | SEQ ID NO. 43 |
| P09/15- | 5′-GGCATACGAGAT-Q-3′ | (length 12) | SEQ ID NO. 44 |
| ASQ | | | |

The combination of oligonucleotides used for our feasibility studies were:

| Tested duplex scorpion | | | | |
|---|---|---|---|---|
| combinations (green if | | P09/15-ASQ | | |
| functional) | | P09-R5 | P15-R5 | P15-R8 |
| P06-ASQ | P06-F5 | | | |
| | P06-F8 | | | |
| P06v4-ASQ | P06v4-F5 | | | |
| | P06v4-F8 | | | |

### FIGURE CAPTIONS AND EXAMPLES

FIG. 1 exemplifies the diversity of Illumina library adapter sequences. Illumina libraries contain conserved P5 and P7 regions and variable regions that differ in length and composition between Illumina library types.

FIG. 2 shows that preferably, the assay consists of 2 primers Fwd and Rev and a set of two 5′-hydrolysis probes labelled with different fluorophores. The set of two probes target the same strand either forward or reverse or opposite strands with primer-probes pairs of for example P5 and P7 each on the same strand. Probes are modified with respective quenchers at the 3′ end. Dark colors indicate the conserved adapter regions and light-colored regions represent the adapter regions that vary in sequence between for example the different Illumina library types.

FIG. 3 shows exemplary target sequences for the novel assay designs. Four representative Illumina libraries were selected for proof-of-concept testing of initial assay designs. The libraries have been prepared using different library preparation kits and differ in the length of the inserted target DNAs; See below. Fragment lengths correspond to library fragments including their linked adapters.

| ID | Type | Average length |
|---|---|---|
| 30170 | 16S rDNA | 794 bp |
| 30186 | Whole transcriptome | 311 bp |
| 30218 | QIAseq miRNA | 165 bp |
| 30130 | QIAseq targeted DNA | 496 bp |

FIG. 4 shows an exemplary assay design that consists of 2 primers and 2 probes. Each probe is labelled with a different fluorophore (FAM and HEX) at the 5′end and a quencher at the 3′end. Primer and probe designs used for the proof-of-concept testing are shown as aligned sequences. Oligo sequences are aligned against a double indexed Illumina TrueSeq target library sequence with adapter regions P5 and P7 highlighted. The primers F4, F5 and F6, and the probes target the same strand. The reverse primer R5 targets the opposite strand. Locked nucleic acids (LNAs) within oligos are highlighted.

FIG. 5 shows two examples of P5/P7-targetting assay designs. In dPCR, both assays produce a clear signal to noise separation in both channels that can be seen in the 2D scatterplots. Further, both assays precisely quantify 3 dilutions of a library test template as shown by the mean concentrations in copies/μl reaction in the bar graph. Shown are 2D scatterplots and quantification bar graphs of two assay designs. Dots in 2D scatterplots depict the green and yellow relative fluorescence of individual partitions of the dPCR reaction with negative signals highlighted in grey and double positive partitions highlighted in dark blue. Each bar in the bar graphs shows mean concentrations in copies/μl of the reaction of 3 replicates each. The expected concentrations of the analyzed test library template is given below the bars. NTC: no template control

FIG. 6 shows experiments done that demonstrate the advantage over the most sophisticated product concepts currently on the market; a comparison to the double probe-based assay from BioRad. In total, 4 Illumina test libraries of different type and length of the target DNA were used for quantification. The 4 test libraries were quantified on both digital PCR systems using one invented assay design on the QIAcuity and the competitor assay from BioRad on the QX200 ddPCR instrument. Assays on the QX200 were run following manufacturer's instructions and using instrument-specific chemistries. In contrast to the Biorad assay that fails to capture the QIAseq libraries the invented assay design captures both TruSeq and QIAseq test libraries. Depicted are 2D scatterplots of both dPCR and ddPCR runs highlighting RFU values of double positive partitions in blue (dPCR) and in orange (ddPCR).

FIG. 7

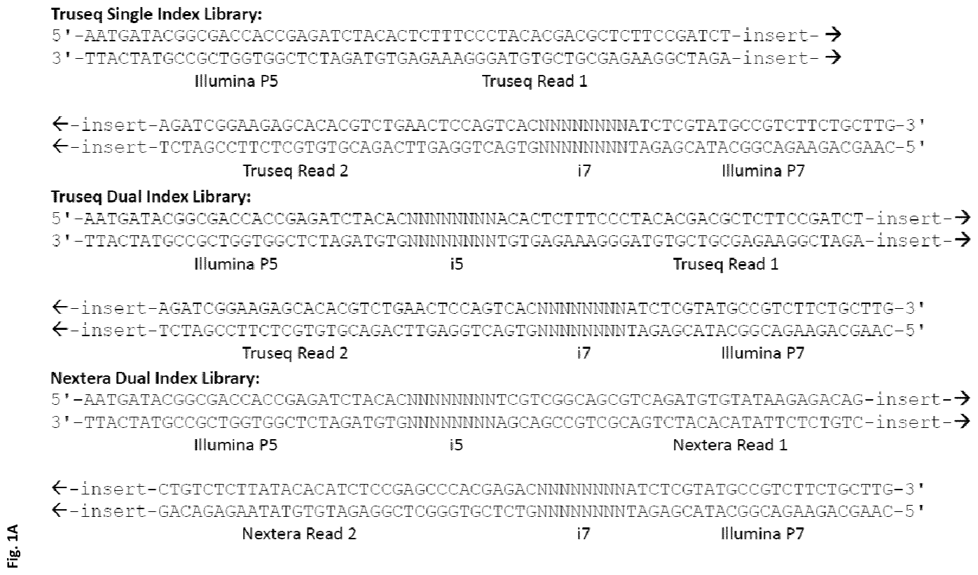

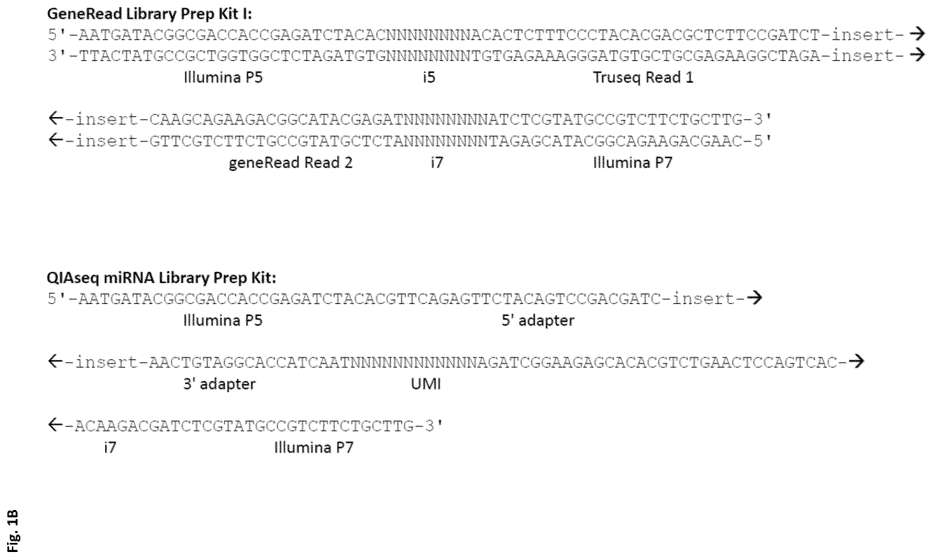

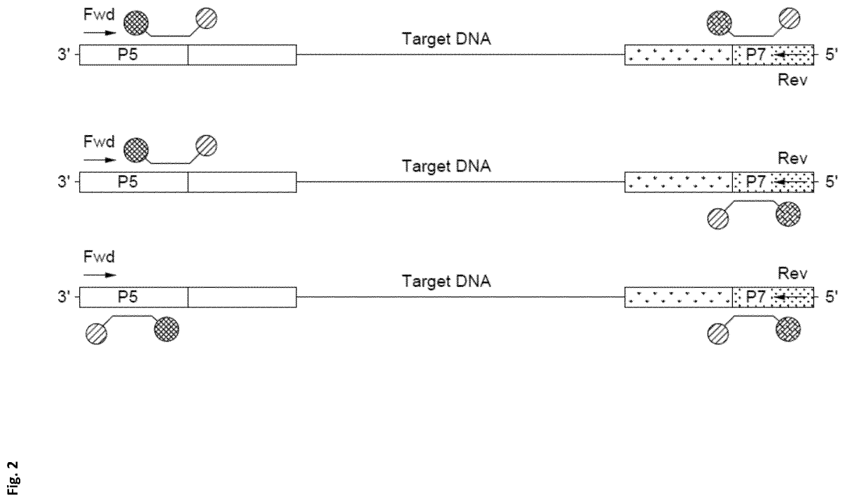

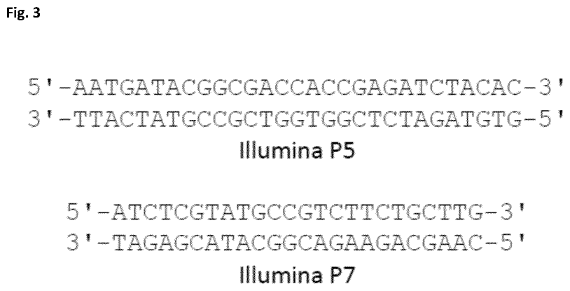

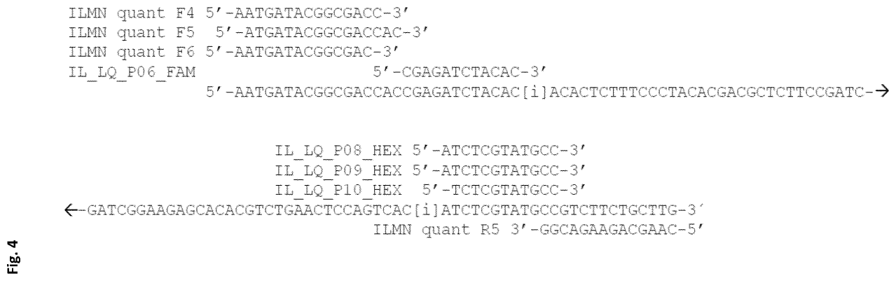

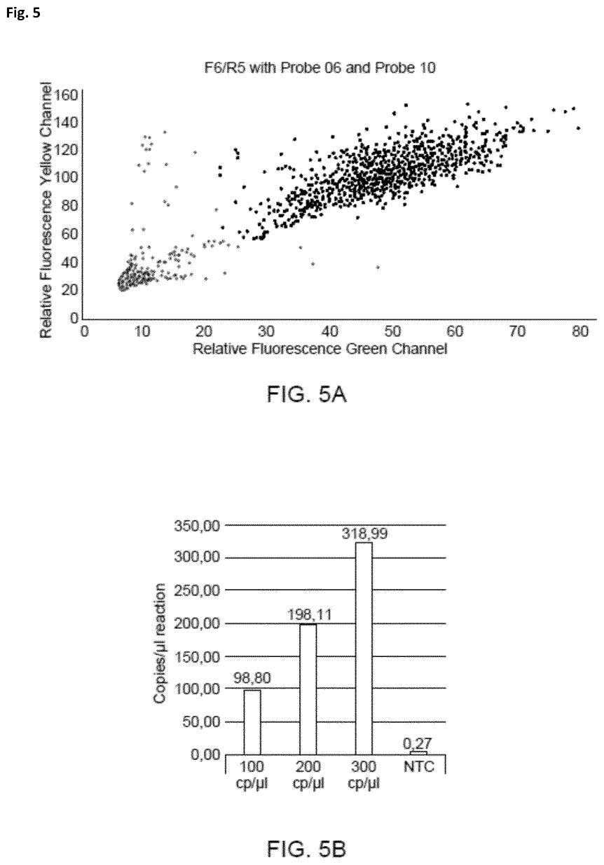

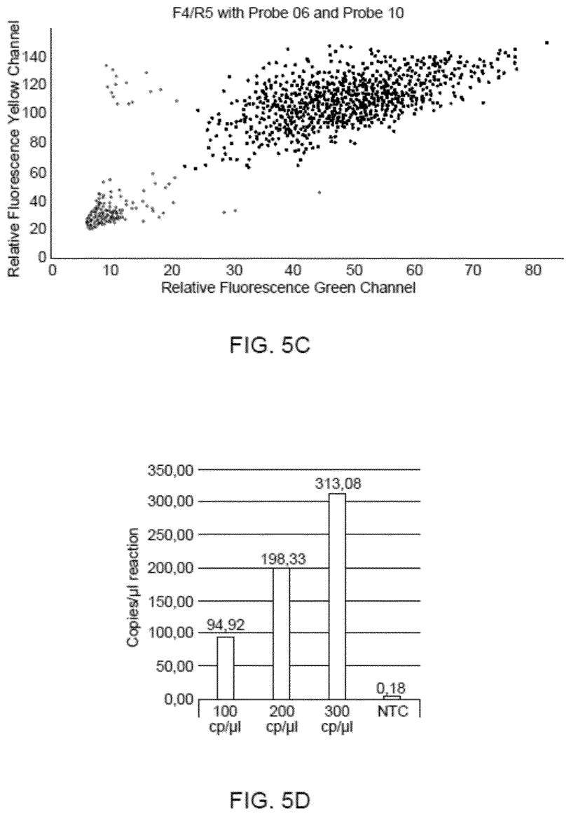

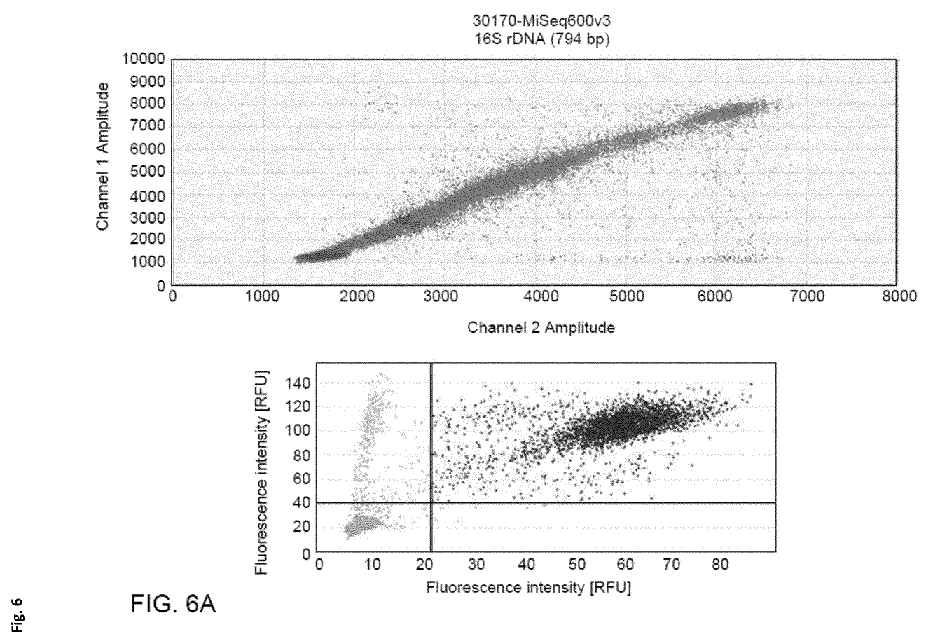

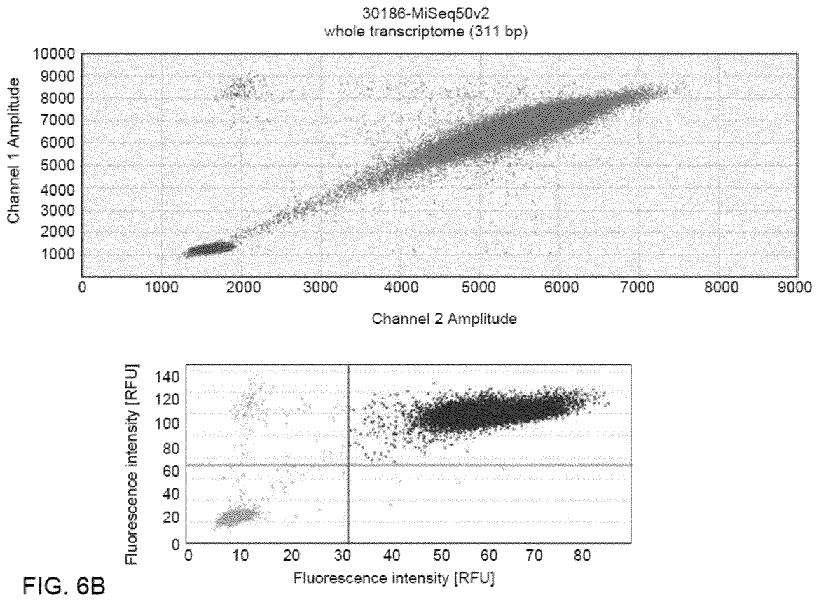

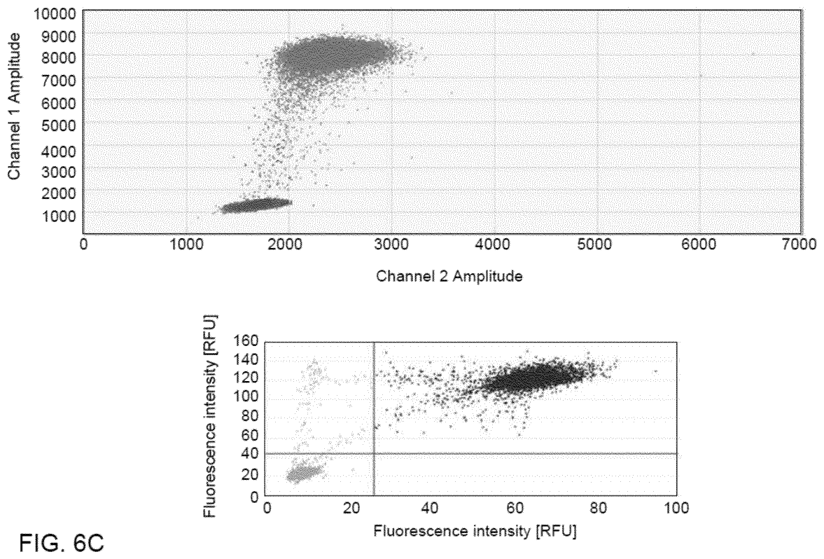

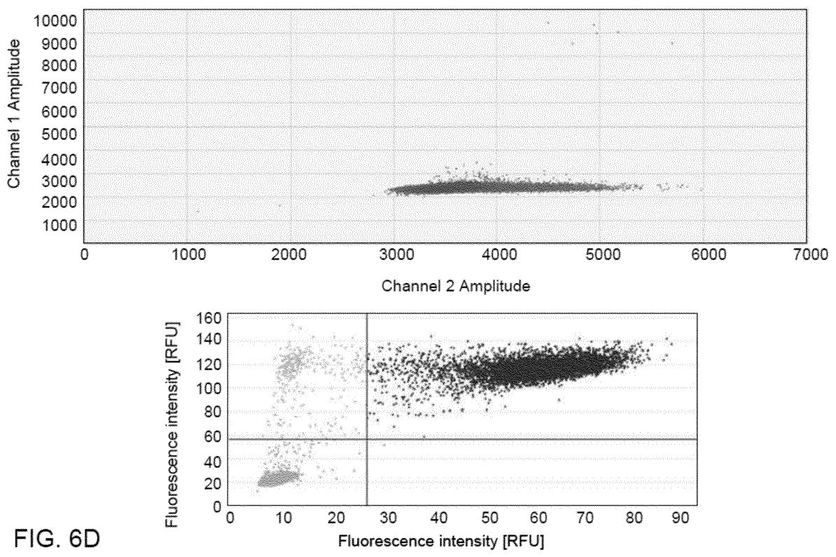

### Materials and Methods

a) Instruments and plastics

The invented assays were tested on the QIAcuity 1, 4 and 8 using 96 well nanoplates (96LV).

### b) Chemicals

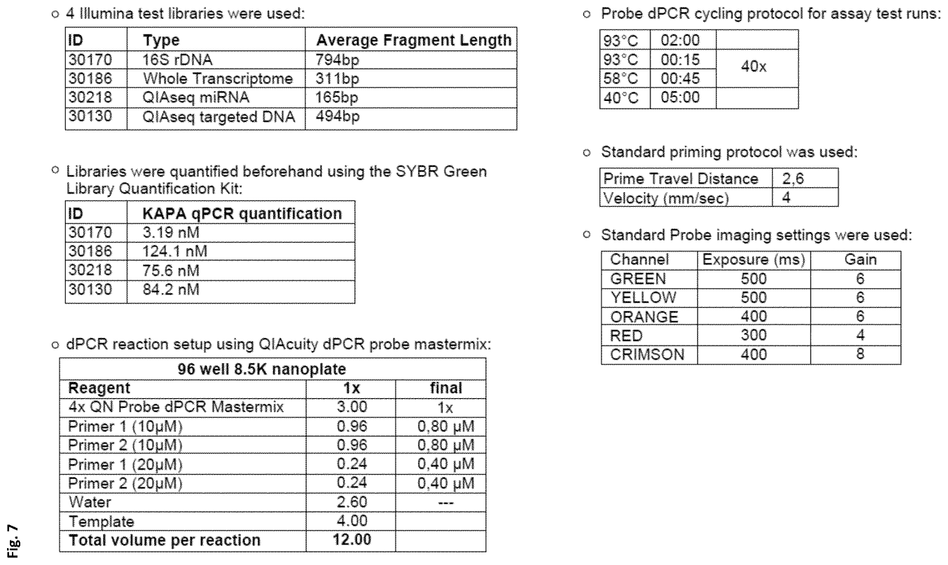

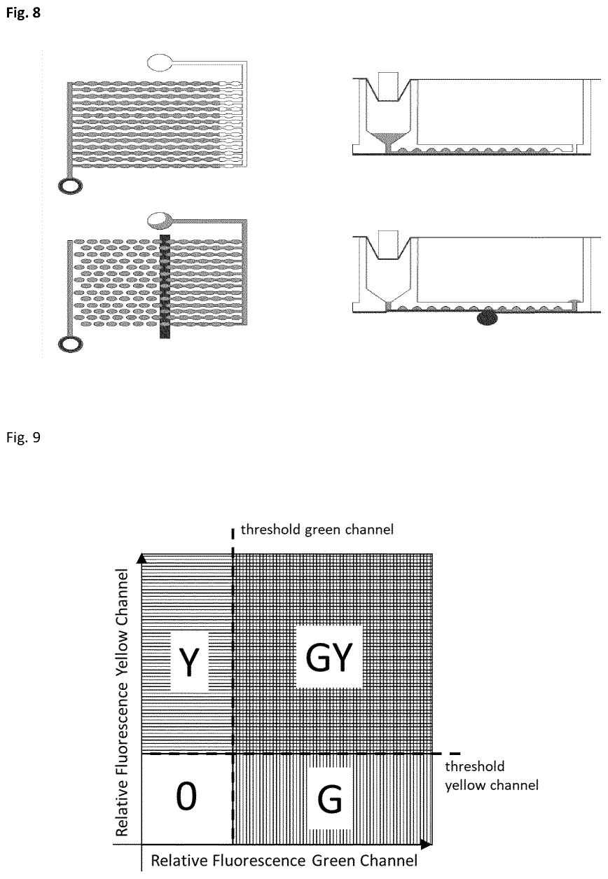

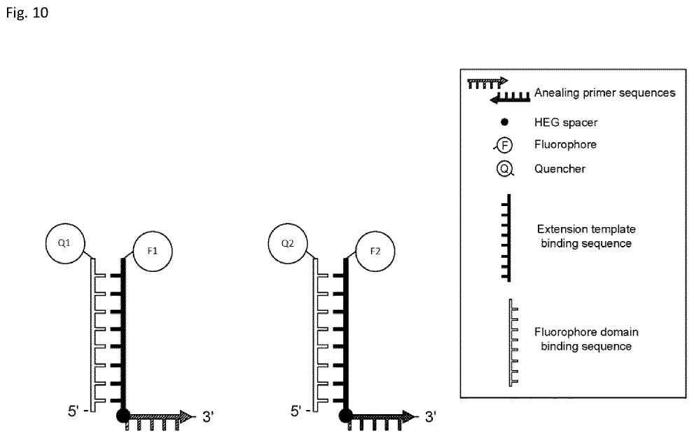

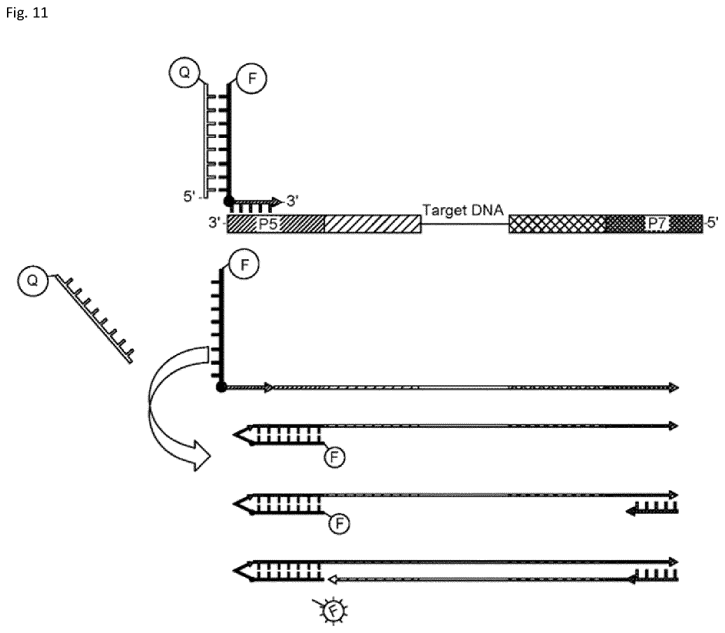

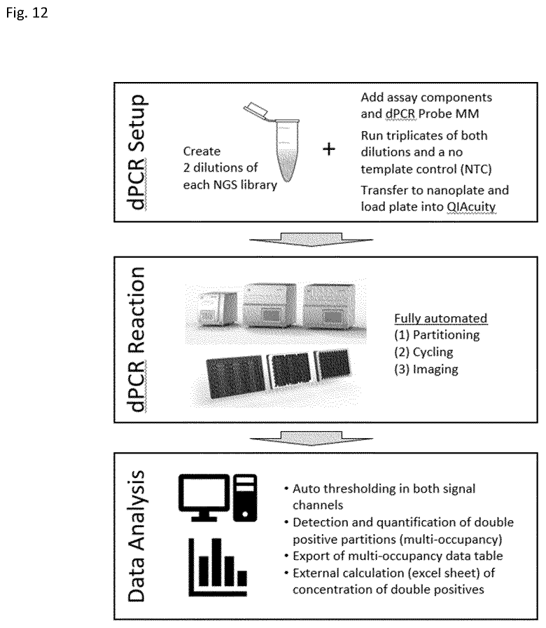

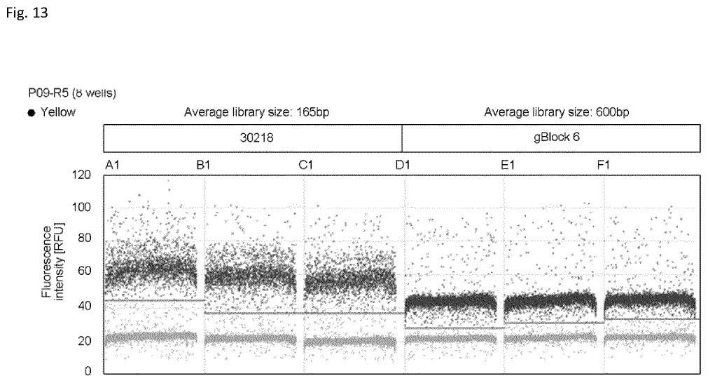

Digital PCR on the QIAcuity used the standard QIAcuity dPCR Probe mastermix. A detailed protocol for setting up the dPCR reaction is given in FIG. 7. Oligos were ordered from Biomers and IDT.

### c) Protocols

A summary of applied protocols is given in FIG. 7.

dPCR Reaction Setup Protocol
1. Thaw the QIAcuity Probe PCR master mix, template DNA, primers, probes, and RNase-free water. Vigorously mix the QIAcuity Probe PCR master mix and the individual solutions. Centrifuge briefly to collect liquids at the bottom of the tubes.
2. Prepare a reaction mix for the number of reactions needed according to Table 2. Due to the hot-start, it is not necessary to keep samples on ice during reaction setup or while programming the QIAcuity instrument.
3. Vortex the reaction mix.
4. Dispense appropriate volumes of the reaction mix, which contains all components except the template, into the wells of a standard PCR plate. Then, add template DNA or cDNA into each well that contains the reaction mix.
5. Transfer the content of each well from the standard PCR plate to the wells of the nanoplate.
6. Seal the nanoplate properly using the QIAcuity Nanoplate Seal provided in the QIAcuity Nanoplate Kits.
7. Sealed plates were placed in a QIAcuity dPCR instrument that automatically performs all subsequent steps (Priming, rolling, cycling and imaging).
8. Image analysis was done using the QIAcuity Software suite.

FIG. 8

Device set-up; See detailed description above.

FIG. 9

Schematic two-dimensional scatterplot of different signal populations. After setting thresholds for the green and yellow channel partitions that are double negative (0), only show green (G) or yellow (Y) signals and partitions that are positive for both channels (GY) localize in 4 distinct areas in the 2D scatterplot.

FIG. 10

The figure shows the preferred embodiment with an assay design based on 2 Duplex Scorpions each consisting of one Fluorophore-primer and one quencher oligo.

FIG. 11

The figure shows the preferred embodiment of Duplex-Scorpion based detection of NGS library fragments exemplary for one of the two adapters. Two duplex scorpions are preferred, each specific to one of the two adapters P5 and P7 (in ILLUMINA NGS libraries)). Specificity to adaptors may be adjusted according to the library to be tested.

FIG. 12

FIG. 12 shows the workflow as develop by the inventors. It is shown the preferred workflow for NGS library quantification with the QIAcuity dPCR system.

FIG. 13

FIG. 13 shows the fragment length-dependent signal, intensity after endpoint dPCR.

1-15. (canceled)

16: A method for examining a nucleic acid amplification product, comprising the steps of
(a) providing an amplification product comprising
(i) an upstream and a downstream primer region and an upstream and a downstream primer binding said upstream and said downstream primer region, respectively,
(b) examining the amplification product by amplifying said amplification product in a PCR reaction, wherein
(i) the upstream and downstream PCR primers in said PCR reaction bind in the upstream and downstream primer region of the amplification product,
(ii) a first and a second oligonucleotide probe are placed downstream of the first and second PCR primers and within the upstream and the downstream primer region,
(c) performing a PCR reaction, preferably a digital PCR reaction, and examining the newly formed amplification product, wherein the upstream and downstream primer regions of step (a)(i) have a length of under 35 nucleotides and over 20 nucleotides.

17: The method according to claim 16, wherein the amplification product provided in step (a) further comprises:
(ii) an amplified target region between said upstream and downstream primer region.

18: The method according to claim 16, wherein the primers are between 10 nucleotides and 22 nucleotides in length and/or the probes are between 8 and 17 nucleotides in length.

19: The method according to claim 16, wherein at least one of the primers comprises one or more locked nucleic acid nucleotides (LNA) or another nucleotide analogue that increase template binding strength.

20: The method according to claim 16, wherein at least one of the probes comprises one or more locked nucleic acid nucleotides (LNA) or another nucleotide analogue that increase template binding strength.

21: The method according to claim 16, wherein all primers and probes comprise LNAs.

22: The method according to claim 16, wherein the one or more primers comprise between 1 and 8 LNA nucleotides.

23: The method according to claim 16, wherein the one or more probes comprise between 2 and 12 LNA nucleotides.

24: The method according to claim 16, wherein the amplification product from step (a)(1) stems from amplifying a nucleic acid library.

25: The method according to claim 16, wherein the library is a sequencing library, and/or wherein the probes are two duplex scorpion probes.

26: A nucleic acid amplification composition comprising:
(a) at least two primers having a length of between 10 and 22 nucleotides, and
(b) at least two oligonucleotide probes having a length of between 8 and 17 nucleotides.

27: The nucleic acid amplification composition according to claim 26, wherein (a) further comprises one or more locked nucleic acid nucleotide analogues or another nucleotide analogue that increase template binding strength.

28: The nucleic acid amplification composition according to claim 26, wherein (b) further comprising one or more locked nucleic acid nucleotide analogues or another nucleotide analogue that increase template binding strength.

29: The nucleic acid amplification composition according to claim 26, wherein the probes are two duplex scorpion probes.

30: A kit comprising:
(a) at least two primers which have a length of between 10 and 22 nucleotides, and
(b) at least two oligonucleotide probes having a length of between 8 and 17 nucleotides.

31: The nucleic acid amplification composition according to claim 30, wherein (a) further comprises one or more locked nucleic acid nucleotide analogues or another nucleotide analogue that increase template binding strength.

32: The nucleic acid amplification composition according to claim 30, wherein (b) further comprising one or more locked nucleic acid nucleotide analogues or another nucleotide analogue that increase template binding strength.

33: The method according to claim 16, wherein the probes are hydrolysis probes, wherein each of the first and second oligonucleotide probes carry a label, and wherein the label on the two probes differs.

34: The method according to claim 33, wherein the nucleic acid amplification product stems from amplifying a nucleic acid library, and wherein the library is a sequencing library.

35: The method according to claim 16, wherein the amplification step (b) is a digital PCR assay, and wherein the template is subdivided into partitions or droplets.


SEQUENCE LISTING (44 sequences)

SEQ ID NO: 1 - DNA, 29 bp, synthetic construct
  misc_feature 1..29: note = P5 Adapter region
  primer_bind 1..29: note = P5adapter
  source 1..29: mol_type = other DNA
aatgatacggcgaccaccgagatctacac

SEQ ID NO: 2 - DNA, 29 bp, synthetic construct
  misc_feature 1..29: note = P5 Adapter region
  primer_bind 1..29: note = P5 adapter
  source 1..29: mol_type = other DNA
gtgtagatctcggtggtcgccgtatcatt

SEQ ID NO: 3 - DNA, 24 bp, synthetic construct
  misc_feature 1..24: note = P7 Adapter region
  primer_bind 1..24: note = P7adapter
  source 1..24: mol_type = other DNA
atctcgtatgccgtcttctgcttg

SEQ ID NO: 4 - DNA, 24 bp, synthetic construct
  misc_feature 1..24: note = P7 Adapter region
  primer_bind 1..24: note = P7adapter
  source 1..24: mol_type = other DNA
caagcagaagacggcatacgagat

SEQ ID NO: 5 - DNA, 124 bp, synthetic construct
  misc_feature 1..124: note = Library sequence
  primer_bind 1..29: note = P5adapter
  misc_recomb 58..59: note = insert insertion site
  misc_feature 93..100: note = Index sequence (variable sequence)
  primer_bind 101..124: note = P7adapter
  source 1..124: mol_type = other DNA
aatgatacggcgaccaccgagatctacactctttccctacacgacgctcttccgatctagatcggaagagcacacgtctgaactccagtcacnnnnnnnnatctcgtatgccgtcttctgcttg

SEQ ID NO: 6 - DNA, 124 bp, synthetic construct
  misc_feature 1..124: note = Library sequence
  primer_bind 1..24: note = P7adapter
  misc_feature 25..32: note = Index sequence (variable sequence)
  misc_recomb 66..67: note = insert insertion site
  primer_bind 96..124: note = P5adapter
  source 1..124: mol_type = other DNA
caagcagaagacggcatacgagatnnnnnnnngtgactggagttcagacgtgtgctcttccgatctagatcggaagagcgtcgtgtagggaaagagtgtagatctcggtggtcgccgtatcatt

SEQ ID NO: 7 - DNA, 136 bp, synthetic construct
  misc_feature 1..136: note = Library sequence
  primer_bind 1..29: note = P5adapter
  misc_feature 30..37: note = Index sequence (variable sequence)
  misc_recomb 70..71: note = insert insertion site
  misc_feature 105..112: note = Index sequence (variable sequence)
  primer_bind 113..136: note = P7adapter
  source 1..136: mol_type = other DNA
aatgatacggcgaccaccgagatctacacnnnnnnnnacactctttccctacacgacgctcttccgatctagatcggaagagcacacgtctgaactccagtcacnnnnnnnnatctcgtatgccgtcttctgcttg

SEQ ID NO: 8 - DNA, 136 bp, synthetic construct
  misc_feature 1..136: note = Library sequence
  primer_bind 1..24: note = P7adapter
  misc_feature 25..32: note = Index sequence (variable sequence)
  misc_recomb 66..67: note = insert insertion site
  misc_feature 100..107: note = Index sequence (variable sequence)
  primer_bind 108..136: note = P5adapter
  source 1..136: mol_type = other DNA
caagcagaagacggcatacgagatnnnnnnnngtgactggagttcagacgtgtgctcttccgatctagatcggaagagcgtcgtgtagggaaagagtgtnnnnnnnngtgtagatctcggtggtcgccgtatcatt

SEQ ID NO: 9 - DNA, 136 bp, synthetic construct
  misc_feature 1..136: note = Library sequence
  primer_bind 1..29: note = P5adapter
  misc_feature 30..37: note = Index sequence (variable sequence)
  misc_recomb 70..71: note = insert insertion site
  misc_feature 105..112: note = Index sequence (variable sequence)
  primer_bind 113..136: note = P7adapter
  source 1..136: mol_type = other DNA
aatgatacggcgaccaccgagatctacacnnnnnnnntcgtcggcagcgtcagatgtgtataagagacagctgtctcttatacacatctccgagcccacgagacnnnnnnnnatctcgtatgccgtcttctgcttg

SEQ ID NO: 10 - DNA, 136 bp, synthetic construct
  misc_feature 1..136: note = Library sequence
  primer_bind 1..24: note = P7adapter
  misc_feature 25..32: note = Index sequence (variable sequence)
  misc_recomb 66..67: note = insert insertion site
  misc_feature 100..107: note = Index sequence (variable sequence)
  primer_bind 108..136: note = P5adapter
  source 1..136: mol_type = other DNA
caagcagaagacggcatacgagatnnnnnnnngtctcgtgggctcggagatgtgtataagagacagctgtctcttatacacatctgacgctgccgacgannnnnnnngtgtagatctcggtggtcgccgtatcatt

SEQ ID NO: 11 - DNA, 122 bp, synthetic construct
  misc_feature 1..122: note = Library sequence
  primer_bind 1..29: note = P5adapter
  misc_recomb 58..59: note = insert insertion site
  misc_feature 93..98: note = Index sequence (variable sequence)
  primer_bind 99..122: note = P7adapter
  source 1..122: mol_type = other DNA
aatgatacggcgaccaccgagatctacactctttccctacacgacgctcttccgatctagatcggaagagcacacgtctgaactccagtcacnnnnnnatctcgtatgccgtcttctgcttg

SEQ ID NO: 12 - DNA, 122 bp, synthetic construct
  misc_feature 1..122: note = Library sequence
  primer_bind 1..24: note = P7adapter
  misc_feature 25..30: note = Index sequence (variable sequence)
  misc_recomb 64..65: note = inser insertion site
  primer_bind 94..122: note = P5adapter
  source 1..122: mol_type = other DNA
caagcagaagacggcatacgagatnnnnnngtgactggagttcagacgtgtgctcttccgatctagatcggaagagcgtcgtgtagggaaagagtgtagatctcggtggtcgccgtatcatt

SEQ ID NO: 13 - DNA, 126 bp, synthetic construct
  misc_feature 1..126: note = Library sequence
  primer_bind 1..29: note = P5adapter
  misc_feature 30..37: note = Index sequence (variable sequence)
  misc_recomb 70..71: note = insert insertion site
  misc_feature 95..102: note = Index sequence (variable sequence)
  primer_bind 103..126: note = P7adapter
  source 1..126: mol_type = other DNA
aatgatacggcgaccaccgagatctacacnnnnnnnnacactctttccctacacgacgctcttccgatctcaagcagaagacggcatacgagatnnnnnnnnatctcgtatgccgtcttctgcttg

SEQ ID NO: 14 - DNA, 126 bp, synthetic construct
  misc_feature 1..126: note = Library sequence
  primer_bind 1..24: note = P7adapter
  misc_feature 25..32: note = Index sequence (variable sequence)
  misc_recomb 56..57: note = insert insertion site
  misc_feature 90..97: note = Index sequence (variable sequence)
  primer_bind 98..126: note = P5adapter
  source 1..126: mol_type = other DNA
caagcagaagacggcatacgagatnnnnnnnnatctcgtatgccgtcttctgcttgagatcggaagagcgtcgtgtagggaaagagtgtnnnnnnnngtgtagatctcggtggtcgccgtatcatt

SEQ ID NO: 15 - DNA, 152 bp, synthetic construct
  misc_feature 1..152: note = Library sequence
  primer_bind 1..29: note = P5adapter
  misc_recomb 55..56: note = insert insertion site
  misc_feature 75..86: note = Index sequence (variable sequence)
  primer_bind 129..152: note = P7adapter
  source 1..152: mol_type = other DNA
aatgatacggcgaccaccgagatctacacgttcagagttctacagtccgacgatcaactgtaggcaccatcaatnnnnnnnnnnnnagatcggaagagcacacgtctgaactccagtcacacaagacgatctcgtatgccgtcttctgcttg

SEQ ID NO: 16 - DNA, 15 bp, synthetic construct
  misc_feature 1..15: note = FW Primer F4
  modified_base 2: mod_base = OTHER; note = LNA
  modified_base 8: mod_base = OTHER; note = LNA
  modified_base 12: mod_base = OTHER; note = LNA
  source 1..15: mol_type = other DNA
aatgatacggcgacc

SEQ ID NO: 17 - DNA, 16 bp, synthetic construct
  misc_feature 1..16: note = FW Primer F5
  modified_base 13: mod_base = OTHER; note = LNA
  source 1..16: mol_type = other DNA
atgatacggcgaccac

SEQ ID NO: 18 - DNA, 14 bp, synthetic construct
  misc_feature 1..14: note = FW Primer F6
  modified_base 3: mod_base = OTHER; note = LNA
  modified_base 6: mod_base = OTHER; note = LNA
  modified_base 9: mod_base = OTHER; note = LNA
  modified_base 13: mod_base = OTHER; note = LNA
  source 1..14: mol_type = other DNA
aatgatacggcgac

SEQ ID NO: 19 - DNA, 14 bp, synthetic construct
  misc_feature 1..14: note = RE Primer R5
  modified_base 3: mod_base = OTHER; note = LNA
  modified_base 7: mod_base = OTHER; note = LNA
  modified_base 13: mod_base = OTHER; note = LNA
  source 1..14: mol_type = other DNA
caagcagaagacgg

SEQ ID NO: 20 - DNA, 12 bp, synthetic construct
  misc_feature 1..12: note = Probe P06
  modified_base 2: mod_base = OTHER; note = LNA
  modified_base 3: mod_base = OTHER; note = LNA
  modified_base 4: mod_base = OTHER; note = LNA
  modified_base 5: mod_base = OTHER; note = LNA
  modified_base 7: mod_base = OTHER; note = LNA
  modified_base 9: mod_base = OTHER; note = LNA
  modified_base 10: mod_base = OTHER; note = LNA
  source 1..12: mol_type = other DNA
cgagatctacac

SEQ ID NO: 21 - DNA, 12 bp, synthetic construct
  misc_feature 1..12: note = Probe P08
  modified_base 3: mod_base = OTHER; note = LNA
  modified_base 5: mod_base = OTHER; note = LNA
  modified_base 6: mod_base = OTHER; note = LNA
  modified_base 7: mod_base = OTHER; note = LNA
  modified_base 9: mod_base = OTHER; note = LNA
  modified_base 10: mod_base = OTHER; note = LNA
  source 1..12: mol_type = other DNA
atctcgtatgcc

SEQ ID NO: 22 - DNA, 12 bp, synthetic construct
  misc_feature 1..12: note = Probe P09
  modified_base 2: mod_base = OTHER; note = LNA
  modified_base 3: mod_base = OTHER; note = LNA
  modified_base 6: mod_base = OTHER; note = LNA
  modified_base 7: mod_base = OTHER; note = LNA
  modified_base 9: mod_base = OTHER; note = LNA
  modified_base 11: mod_base = OTHER; note = LNA
  source 1..12: mol_type = other DNA
atctcgtatgcc

SEQ ID NO: 23 - DNA, 11 bp, synthetic construct
  misc_feature 1..11: note = Probe P10
  modified_base 2: mod_base = OTHER; note = LNA
  modified_base 3: mod_base = OTHER; note = LNA
  modified_base 4: mod_base = OTHER; note = LNA
  modified_base 5: mod_base = OTHER; note = LNA
  modified_base 8: mod_base = OTHER; note = LNA
  modified_base 10: mod_base = OTHER; note = LNA
  source 1..11: mol_type = other DNA
tctcgtatgcc

SEQ ID NO: 24 - DNA, 118 bp, synthetic construct
  misc_feature 1..118: note = Library sequence
  primer_bind 1..29: note = P5adapter
  primer_bind 95..118: note = P7adapter
  source 1..118: mol_type = other DNA
aatgatacggcgaccaccgagatctacacacactctttccctacacgacgctcttccgatcgatcggaagagcacacgtctgaactccagtcacatctcgtatgccgtcttctgcttg

SEQ ID NO: 25 - DNA, 12 bp, synthetic construct
  misc_feature 1: note = FAM-G (FAM dye 5' of G)
  modified_base 3..4: mod_base = OTHER; note = LNA
  modified_base 6: mod_base = OTHER; note = LNA
  modified_base 8..11: mod_base = OTHER; note = LNA
  misc_feature 12: note = G-HEG (one hexethylene glycol spacer 3' of G that links the last nucleotide of SEQ ID NO. 25 to the first nucleotide of SEQ ID NO. 26)
  source 1..12: mol_type = other DNA
gtgtagatctcg

SEQ ID NO: 26 - DNA, 16 bp, synthetic construct
  source 1..16: mol_type = other DNA
  misc_feature 1: note = HEG-A (one hexethylene glycol spacer 5' of A that links the last nucleotide of SEQ ID NO. 25 to the first nucleotide of SEQ ID NO. 26)
  modified_base 13: mod_base = OTHER; note = LNA
atgatacggcgaccac

SEQ ID NO: 27 - DNA, 12 bp, synthetic construct
  misc_feature 12: note = C-Q (Quencher 3' of C)
  source 1..12: mol_type = other DNA
cgagatctacac

SEQ ID NO: 28 - DNA, 11 bp, synthetic construct
  misc_feature 1: note = FAM-T (FAM dye 5' of T)
  modified_base 2..3: mod_base = OTHER; note = LNA
  modified_base 5: mod_base = OTHER; note = LNA
  modified_base 7..10: mod_base = OTHER; note = LNA
  source 1..11: mol_type = other DNA
  misc_feature 11: note = G-HEG (one hexethylene glycol spacer 3' of G that links the last nucleotide of SEQ ID NO. 28 to the first nucleotide of SEQ ID NO. 29)
tgtagatctcg

SEQ ID NO: 29 - DNA, 16 bp, synthetic construct
  source 1..16: mol_type = other DNA
  misc_feature 1: note = HEG-A (one hexethylene glycol spacer 5' of A that links the last nucleotide of SEQ ID NO. 28 to the first nucleotide of SEQ ID NO. 29)
  modified_base 13: mod_base = OTHER; note = LNA
atgatacggcgaccac

SEQ ID NO: 30 - DNA, 11 bp, synthetic construct
  misc_feature 11: note = A-Q (Quencher 3' of A)
  source 1..11: mol_type = other DNA
cgagatctaca

SEQ ID NO: 31 - DNA, 12 bp, synthetic construct
  misc_feature 1: note = HEX-A (HEX dye 5' of A)
  modified_base 2..3: mod_base = OTHER; note = LNA
  modified_base 6..7: mod_base = OTHER; note = LNA
  modified_base 9: mod_base = OTHER; note = LNA
  modified_base 11: mod_base = OTHER; note = LNA
  misc_feature 12: note = C-HEG (one hexethylene glycol spacer 3' of C that links the last nucleotide of SEQ ID NO. 31 to the first nucleotide of SEQ ID NO. 32)
  source 1..12: mol_type = other DNA
atctcgtatgcc

SEQ ID NO: 32 - DNA, 14 bp, synthetic construct
  source 1..14: mol_type = other DNA
  misc_feature 1: note = HEG-C (one hexethylene glycol spacer 5' of C that links the last nucleotide of SEQ ID NO. 31 to the first nucleotide of SEQ ID NO. 32)
  modified_base 3: mod_base = OTHER; note = LNA
  modified_base 7: mod_base = OTHER; note = LNA
  modified_base 13: mod_base = OTHER; note = LNA
caagcagaagacgg

SEQ ID NO: 33 - DNA, 12 bp, synthetic construct
  misc_feature 1: note = HEX-A (HEX dye 5' of A)
  modified_base 2..3: mod_base = OTHER; note = LNA
  modified_base 5..6: mod_base = OTHER; note = LNA
  modified_base 8: mod_base = OTHER; note = LNA
  modified_base 10: mod_base = OTHER; note = LNA
  misc_feature 12: note = C-HEG (one hexethylene glycol spacer 3' of C that links the last nucleotide of SEQ ID NO. 33 to the first nucleotide of SEQ ID NO. 34)
  source 1..12: mol_type = other DNA
atctcgtatgcc

SEQ ID NO: 34 - DNA, 14 bp, synthetic construct
  source 1..14: mol_type = other DNA
  misc_feature 1: note = HEG-C (one hexethylene glycol spacer 5' of C that links the last nucleotide of SEQ ID NO. 33 to the first nucleotide of SEQ ID NO. 34)
  modified_base 3: mod_base = OTHER; note = LNA
  modified_base 7: mod_base = OTHER; note = LNA
  modified_base 13: mod_base = OTHER; note = LNA
caagcagaagacgg

SEQ ID NO: 35 - DNA, 12 bp, synthetic construct
  misc_feature 12: note = T-Q (Quencher 3' of T)
  source 1..12: mol_type = other DNA
ggcatacgagat

SEQ ID NO: 36 - DNA, 12 bp, synthetic construct
  misc_feature 1: note = FAM-G (FAM dye 5' of G)
  modified_base 3..4: mod_base = OTHER; note = LNA
  modified_base 6: mod_base = OTHER; note = LNA
  modified_base 8..11: mod_base = OTHER; note = LNA
  misc_feature 12: note = G-HEG (one hexethylene glycol spacer 3' of G that links the last nucleotide of SEQ ID NO. 36 to the first nucleotide of SEQ ID NO. 37)
  source 1..12: mol_type = other DNA
gtgtagatctcg

SEQ ID NO: 37 - DNA, 18 bp, synthetic construct
  source 1..18: mol_type = other DNA
  misc_feature 1: note = HEG-A (one hexethylene glycol spacer 5' of A that links the last nucleotide of SEQ ID NO. 36 to the first nucleotide of SEQ ID NO. 37)
agctaatgatacggcgac

SEQ ID NO: 38 - DNA, 12 bp, synthetic construct
  misc_feature 12: note = C-Q (Quencher 3' of C)
  source 1..12: mol_type = other DNA
cgagatctacac

SEQ ID NO: 39 - DNA, 11 bp, synthetic construct
  misc_feature 1: note = FAM-T (FAM dye 5' of T)
  modified_base 2..3: mod_base = OTHER; note = LNA
  modified_base 5: mod_base = OTHER; note = LNA
  modified_base 7..10: mod_base = OTHER; note = LNA
  misc_feature 11: note = G-HEG (one hexethylene glycol spacer 3' of G that links the last nucleotide of SEQ ID NO. 39 to the first nucleotide of SEQ ID NO. 40)
  source 1..11: mol_type = other DNA
tgtagatctcg

SEQ ID NO: 40 - DNA, 18 bp, synthetic construct
  source 1..18: mol_type = other DNA
  misc_feature 1: note = HEG-A (one hexethylene glycol spacer 5' of A that links the last nucleotide of SEQ ID NO. 39 to the first nucleotide of SEQ ID NO. 40)
agctaatgatacggcgac

SEQ ID NO: 41 - DNA, 11 bp, synthetic construct
  misc_feature 11: note = A-Q (Quencher 3' of A)
  source 1..11: mol_type = other DNA
cgagatctaca

SEQ ID NO: 42 - DNA, 12 bp, synthetic construct
  misc_feature 1: note = HEX-A (HEX dye 5' of A)
  modified_base 2..3: mod_base = OTHER; note = LNA
  modified_base 5..6: mod_base = OTHER; note = LNA
  modified_base 8: mod_base = OTHER; note = LNA
  modified_base 10: mod_base = OTHER; note = LNA
  misc_feature 12: note = C-HEG (one hexethylene glycol spacer 3' of C that links the last nucleotide of SEQ ID NO. 42 to the first nucleotide of SEQ ID NO. 43)
  source 1..12: mol_type = other DNA
atctcgtatgcc

SEQ ID NO: 43 - DNA, 16 bp, synthetic construct
  source 1..16: mol_type = other DNA
  misc_feature 1: note = HEG-A (one hexethylene glycol spacer 5' of A that links the last nucleotide of SEQ ID NO. 42 to the first nucleotide of SEQ ID NO. 43)
  modified_base 7: mod_base = OTHER; note = LNA
  modified_base 11: mod_base = OTHER; note = LNA
agctcaagcagaagac

SEQ ID NO: 44 - DNA, 12 bp, synthetic construct
  misc_feature 12: note = T-Q (Quencher 3' of T)
  source 1..12: mol_type = other DNA
ggcatacgagat